(19) United States
(12) Patent Application Publication
(10) Pub. No.: US 2026/0109666 A1
(43) Pub. Date: Apr. 23, 2026

(54) ACETONITRILE COMPOSITIONS AND METHODS OF MAKING THE SAME

(71) Applicant: Safion Renewables Inc., Louisville, CO (US)

(72) Inventors: Eric M. Karp, Arvada, CO (US); Jacob Scott Kruger, Arvada, CO (US); Ryan Prestangen, Denver, CO (US); Lauren Ann Riley Drumm, Lafayette, CO (US); Andrew John Borchert, Arvada, CO (US); Alexander Hunt Jenkins, Louisville, CO (US)

(21) Appl. No.: 19/360,834

(22) Filed: Oct. 16, 2025

Related U.S. Application Data
(60) Provisional application No. 63/708,557, filed on Oct. 17, 2024.

(51) Int. Cl. C07C 255/04; C07C 253/22; C07C 253/34; C12P 7/40

(52) U.S. Cl. CPC C07C 255/04; C07C 253/22; C07C 253/34; C12P 7/40

(57) ABSTRACT

Provided herein are methods of making acetonitrile from biologically produced precursors. The methods herein may be less carbon intensive and produce fewer toxic byproducts than traditional methods. Also provided herein are acetonitrile compositions, such as prepared by the methods provided herein.

This application claims the benefit of U.S. Provisional Patent Application Ser. No. 63/708,557 entitled “ACETONITRILE COMPOSITIONS AND METHODS OF MAKING THE SAME,” filed Oct. 17, 2024, the disclosure of which is incorporated herein its entirety by reference.

### BACKGROUND

Acetonitrile is an organic solvent used in analytical chemistry, industrial, and pharmaceutical manufacturing processes. Historically, acetonitrile has been made on the industrial scale as a byproduct of acrylonitrile production (via the Standard Oil of Ohio (SOHIO) process). The global acetonitrile market stood at approximately 180,000 tons in 2022 and is anticipated to continue growing.

### SUMMARY

Discussed herein are methods of making an acetonitrile composition, and acetonitrile compositions.

In a first embodiment, a method of making an acetonitrile composition can include providing a biologically produced acetonitrile precursor, purifying the biologically produced acetonitrile precursor, reacting the biologically produced acetonitrile precursor with a nitrogen source and a catalyst to provide crude acetonitrile, and purifying the crude acetonitrile to provide the acetonitrile composition. In some examples, the reacting produces no more than 20 wt % of organic products other than acetonitrile. In some examples, the acetonitrile composition comprises less than 2 wt % of impurities.

In a second embodiment, a method of making an acetonitrile composition can include providing a biologically produced acetonitrile precursor, purifying the biologically produced acetonitrile precursor, reacting the biologically produced acetonitrile precursor with a nitrogen source and a catalyst to provide crude acetonitrile, and purifying the crude acetonitrile to provide the acetonitrile composition. In some examples, the method does not generate hydrogen cyanide.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 shows an illustrative method for preparing acetonitrile as described herein. In FIG. 1, in some embodiments, each of (102), (103), (104), and (105) may be required. In some instances, (102) may be optional. In some instances, (103) may be optional. In some instances, (104) may be optional. In some instances, (105) may be optional. In some instances, (107) may be optional. In some instances, (112) may be optional.

FIG. 2 shows an illustrative method for preparing acetonitrile as described herein. In FIG. 2, each of (203), (204), and (205) may be required. In some instances, (203) may be optional. In some instances, (204) may be optional. In some instances, (205) may be optional. In some instances, (211) may be optional.

FIG. 3 shows an exemplary reaction mechanism for preparation of acetonitrile from acetic acid.

FIG. 4 shows an illustrative method for purifying acetonitrile as described herein. In FIG. 4, (401) may be optional. In FIG. 4, (402) may be optional. In FIG. 4, in some embodiments, both (401) and (402) may be required.

FIG. 5 shows illustrative procedures for production of acetic acid from fermentation of ethanol.

FIG. 6 shows an illustrative procedure for purifying acetic acid as described herein.

FIG. 7 depicts a chart exhibiting the results of the Examples discussed herein.

FIG. 8 depicts a chart exhibiting the results of the Examples discussed herein.

FIG. 9 depicts a chart exhibiting the results of the Examples discussed herein.

FIG. 10 depicts a chart exhibiting the results of the Examples discussed herein.

FIG. 11 depicts a chart exhibiting the results of the Examples discussed herein.

FIG. 12 depicts a chart exhibiting the results of the Examples discussed herein.

FIG. 13 depicts a chart exhibiting the results of the Examples discussed herein.

FIG. 14 depicts a chart exhibiting the results of the Examples discussed herein.

FIG. 15 depicts a chart exhibiting the results of the Examples discussed herein.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

The present disclosure provides renewable and environmentally friendly methods for producing high-purity acetonitrile using bio-based feedstocks as an alternative to the traditional SOHIO (Standard Oil of Ohio) process. The SOHIO process relies on the use of fossil fuel-based feedstocks and results in the release of toxic side products. Alternative processes to produce high purity acetonitrile that are renewable, more environmentally friendly, and less toxic are needed.

The discussed methods are for bio-based production using biologically produced acetonitrile precursors (primarily acetic acid from fermentation). Further, the methods discussed herein eliminate or reduce toxic or unwanted byproducts. For example, the method does not generate hydrogen cyanide, unlike traditional processes. Moreover, the methods discussed herein give a high purity output. These methods can produce acetonitrile compositions with, for example, less than 2 wt % impurities. Overall, the methods discussed herein can reduce carbon intensity, such as by an approximately 90% reduction in carbon intensity compared to the SOHIO process.

In one example of the methods discussed herein, the method can include steps of biological production, purification, reaction, and final purification. At biological production, an acetonitrile precursor (e.g., acetic acid) can be formed via fermentation. Next, at purification, the precursor can be purified using a variety of methods, such as liquid-liquid extraction and/or distillation. Subsequently, at reaction, a nitrogen source, such as ammonia, and a catalyst can be reacted with the purified precursor, such as at an appropriate reactor temperature and pressure range. During final purification, through steps such as, but not limited to, distillation, oxidation, and polishing, the high purity acetonitrile can be produced.

The discussed methods and compositions provide a variety of advantages over previous methods, some of which are unexpected. More specifically, the disclosed methods for producing acetonitrile from biologically produced precursors offer significant advantages over traditional production methods, particularly the SOHIO process. These benefits include, but are not limited to environmental and sustainability benefits, safety and toxicity advantages, product quality benefits, process and operational advantages, and economic and supply chain benefits.

The methods herein provide a variety of sustainability and environmental benefits. For example, the bio-based acetonitrile production method provides approximately 90% reduction in carbon intensity compared to acetonitrile produced using the traditional SOHIO process. This dramatic improvement in environmental footprint makes the process much more sustainable. Additionally, the methods can utilize entirely biological feedstocks, reducing dependence on fossil fuel-based sources like propylene. The process can use bio-based precursors produced through fermentation, creating a renewable production pathway.

The methods herein provide several toxicity and safety advantages. For example, unlike the SOHIO process, these methods do not generate hydrogen cyanide, a highly toxic byproduct. This can create a safer working environment and reduce environmental contamination risks. Moreover, the process does not require oxygen usage, eliminating an explosive hazard present in the traditional SOHIO process. Additionally, the process involves an endothermic reaction, eliminating the hazard of thermal runaway present in the traditional SOHIO process.

The methods herein provide product quality benefits, for example, a high purity output. The methods herein can produce acetonitrile compositions with less than 2 wt % impurities, less than 1 wt % impurities, or less than 0.5 wt % impurities, suitable for demanding applications including: oligonucleotide synthesis grade quality or pharma grade quality acetonitrile. The resulting acetonitrile can meet strict absorbance specifications across multiple wavelengths (200-400 nm), with very low absorbance indicating high optical purity. Finally, the process can produce anhydrous acetonitrile compositions with less than 20 ppm water, and even less than 10 ppm water in some embodiments.

The methods herein provide several process and operational advantages. Unlike the SOHIO process where acetonitrile is merely a byproduct of acrylonitrile production, these methods are specifically designed for direct preparation of acetonitrile. This allows for process independence; the methods are decoupled from production of other compounds (such as acrylonitrile), providing more flexibility and control over acetonitrile production. The methods herein allow for high selectivity: the reacting step produces no more than 20 wt % of organic products other than acetonitrile, and in some embodiments no more than 10 wt % of organic products other than acetonitrile, indicating high process selectivity. Moreover, the methods can produce acetonitrile in quantities of at least 100 L, and in some cases at least 150 L, making them suitable for industrial-scale production.

Finally, the methods herein allow for economic and supply chain benefits. For example, the process can include provisions for recycling unreacted nitrogen sources (such as ammonia) and water/ethanol streams, improving process economics, e.g., for resource recycling. Moreover, the use of bio-based precursors like acetic acid from fermentation leverages existing biotechnology infrastructure and established fermentation processes. By using renewable biological feedstocks rather than petroleum-derived materials, the process provides greater supply chain security and reduced exposure to fossil fuel price volatility.

These advantages collectively position the disclosed bio-based acetonitrile production methods as a superior alternative to traditional petrochemical processes, offering environmental sustainability, enhanced safety, high product quality, and operational flexibility while maintaining the ability to produce pharmaceutical and oligonucleotide synthesis grade acetonitrile at industrial scales.

### Definitions

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. A comma can be used as a delimiter or digit group separator to the left or right of a decimal mark; for example, “0.000,1” is equivalent to “0.0001.” All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “solvent” as used herein refers to a substance that can dissolve a solid, liquid, or gas. Non-limiting examples of solvents are silicones, organic compounds, water, alcohols, ionic liquids, and supercritical fluids.

As used herein, “pure” or “purity” refer to a substance that is free from or comprises less than a given level or amount of other substances or impurities. For example, the substance can have e.g., 5 wt % or less (e.g., 4 wt %, 3 wt %, 2.5 wt %, 2 wt %, 1.5 wt % 1 wt %, 0.5 wt %, 0.1 wt %, or 0.05 wt %) of total impurities or related substances. “Percent pure” or “percent purity” as used herein refers to a substance comprising less than a given percentage (e.g., wt %, vol %, mol %) of impurities or related substances. For instance, a substance that is 95% pure may refer to the substance comprising less than 5% of impurities or related substances. Methods for determining purity may include, but are not limited to, gas chromatography, UV-V is spectroscopy, FTIR spectroscopy, NMR spectroscopy, and Karl Fischer titration.

### Methods of Making Acetonitrile

Acetonitrile is a solvent often used in analytical chemistry and many manufacturing processes. It is historically produced from the propylene ammoxidation process (also known as the SOHIO process). In the SOHIO process, propylene, ammonia, and oxygen are reacted at high temperature to produce acrylonitrile, where acetonitrile is produced as a byproduct. There are several disadvantages to the currently used SOHIO process in that (1) it requires the use of fossil fuel sources (e.g., propylene), (2) acetonitrile is a byproduct of the SOHIO process, (3) the process is very carbon intensive, (4) the reaction produces toxic side products, including hydrogen cyanide, (5) the reaction requires the use of oxygen which represents an explosive hazard, (6) the reaction is exothermic which creates risk of thermal runaway, and (7) high purity grade acetonitrile is difficult to achieve using the SOHIO process.

Provided herein, in some embodiments, are methods of making acetonitrile (or compositions comprising acetonitrile), also referred to herein as “bio-based acetonitrile.” The methods provided herein are alternatives to the standard propylene ammoxidation process, which allow for preparation of acetonitrile at high levels of purity (e.g., oligonucleotide synthesis quality or pharma grade quality). In some embodiments, the methods herein provide for the use of alternative feedstocks, such as ethanol and sugars, which obviate the need for fossil fuel-based sources (e.g., propylene). For instance, the methods herein provide for the production of (e.g., high purity) acetonitrile by use of 100% bio-based feedstocks. The methods provided herein may be less carbon intensive as a result, allowing for a more ecologically sustainable method for preparation of acetonitrile. Additionally, the methods herein are decoupled from production of other reagents (e.g., acrylonitrile) and provide for direct preparation of acetonitrile that does not require the use of explosive hazards or result in the release of toxic byproduct, such as hydrogen cyanide.

The acetonitrile (e.g., bio-based acetonitrile) composition provided herein may comprise a reduction in carbon intensity (CI) compared to acetonitrile produced using traditional fossil-fuel based methods, such as the SOHIO process. In some embodiments, the acetonitrile (e.g., bio-based acetonitrile) composition comprises an at least 50%, 60%, 70%, 80%, 85%, 86%, 87%, 88%, 89%, 90%, 91%, 92%, 93%, 94%, 95%, 96%, 97%, 98%, 99%, 99.5%, or 100% reduction in CI as compared to an acetonitrile composition produced using traditional fossil-fuel based methods, such as the SOHIO process. In some embodiments, the acetonitrile (e.g., bio-based acetonitrile) composition comprises a reduction in CI greater than 100% (e.g. when the CI is a negative number) as compared to an acetonitrile composition produced using traditional fossil-fuel based methods, such as the SOHIO process. In some embodiments, the acetonitrile (e.g., bio-based acetonitrile) composition comprises an at least 90% reduction in CI as compared to an acetonitrile composition produced using traditional fossil-fuel based methods, such as the SOHIO process. In some embodiments, the acetonitrile (e.g., bio-based acetonitrile) composition comprises an about 90% reduction in CI as compared to an acetonitrile produced composition using the SOHIO process. In some embodiments, CI is measured using the R&D GREET model (e.g., as described at http://greet.anl.gov/).

Carbon Intensity (CI), as described herein, can refer to a measure of carbon dioxide and other greenhouse gases emitted per unit of product produced (e.g., manufacture of acetonitrile). In some embodiments, CI is measured using the The Greenhouse gases, Regulated Emissions, and Energy use in Technologies (GREET) Model, which uses input data related to the lifecycle of various feedstocks and chemicals as well as emissions associated with different manufacturing processes and outputs the calculated CI, determined by assessing the total greenhouse gas emissions produced per unit of product produced (e.g., as described at http://greet.anl.gov/). In some embodiments, CI is measured using the R&D GREET model as described at http://greet.anl.gov/.

In some embodiments, the methods provided herein comprise providing an acetonitrile precursor. In some embodiments, the acetonitrile precursor is acetic acid, an acetate salt (e.g., sodium acetate, potassium acetate, calcium acetate, ammonium acetate, zinc acetate, copper acetate, manganese acetate, iron acetate, magnesium acetate, aluminum acetate, lithium acetate, barium acetate, cobalt acetate, lead acetate, nickel acetate, silver acetate, chromium acetate, chromium acetate, cadmium acetate, or strontium acetate), acetamide, acetaldehyde, acetic anhydride, a substituted or unsubstituted C₂ carbonyl (e.g., ketone or carboxylic acid), or a combination thereof. In some embodiments, the acetonitrile precursor is an acetate salt. In some embodiments, the acetonitrile precursor is acetamide. In some embodiments, the acetonitrile precursor is acetaldehyde. In some embodiments, the acetonitrile precursor is acetic anhydride. In specific embodiments, the acetonitrile precursor is acetic acid.

In some embodiments, the acetonitrile precursor is produced from a biological source (e.g., a biologically produced acetonitrile precursor). The biologically produced acetonitrile precursor may be produced by any suitable method.

In some embodiments, the acetonitrile precursor is provided via catalytic oxidation. In some embodiments, the acetonitrile precursor is acetic acid. In some embodiments, the acetonitrile precursor is acetic acid and is provided via catalytic oxidation of ethanol to acetic acid. In some embodiments, catalytic oxidation of ethanol to acetic acid comprises conversion of ethanol to acetaldehyde. In some embodiments, the catalytic oxidation of ethanol to acetic acid comprises reaction of acetaldehyde to form the acetic acid.

In some embodiments, the methods provided herein incorporate no fossil-derived material into the acetonitrile. For instance, the methods provided herein do not comprise the use of propylene, propane, or any combination thereof.

In some embodiments, the methods comprise use of an acetonitrile precursor comprising at least 80 wt % (e.g., at least 90 wt %, 95 wt %, 99 wt %) of a biologically produced acetonitrile precursor (e.g., acetic acid). In some embodiments, the methods comprise use of an acetonitrile precursor comprising at least 80 wt % of a biologically produced acetonitrile precursor (e.g., acetic acid). In some embodiments, the methods comprise use of an acetonitrile precursor comprising at least 95 wt % of a biologically produced acetonitrile precursor (e.g., acetic acid). In some embodiments, the methods comprise use of an acetonitrile precursor comprising 100 wt % of a biologically produced acetonitrile precursor (e.g., acetic acid), such as biologically produced via the methods described herein.

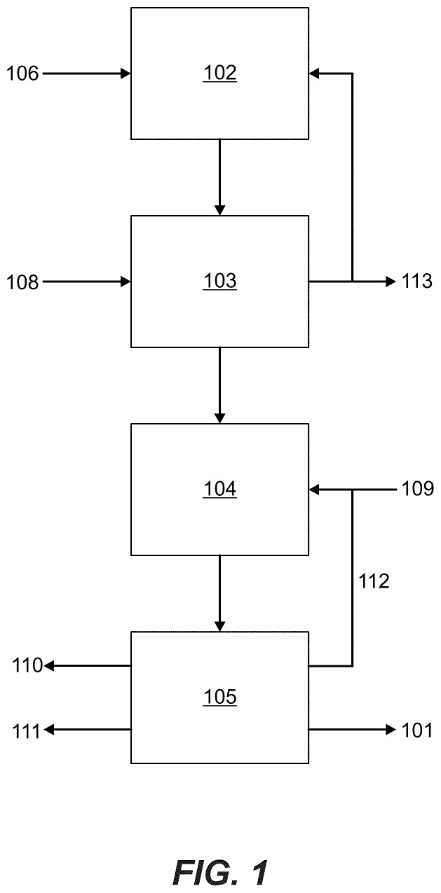

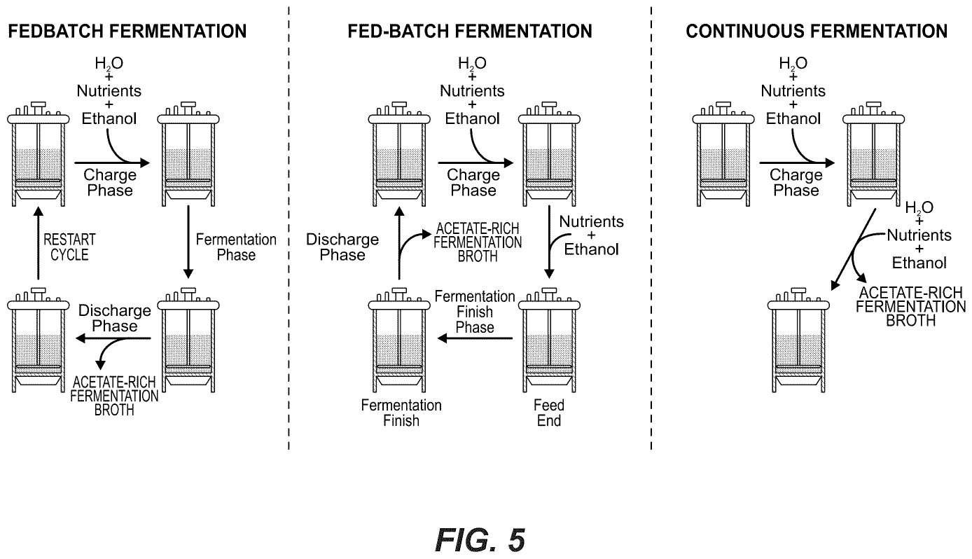

In some embodiments, the biologically produced acetonitrile precursor (e.g., acetic acid) is produced by fermentation. In some embodiments, the biologically produced acetonitrile precursor (e.g., acetic acid) is produced by fermentation, such as described in Example 1 and Example 2, elsewhere herein. In some embodiments, the biologically produced acetonitrile precursor (e.g., acetic acid) is produced by fermentation, such as shown in FIG. 1 (102), or alternatively shown in FIG. 5. In some embodiments, the fermentation is aerobic fermentation. In some embodiments, the fermentation is anaerobic fermentation. In some embodiments, the biologically produced acetonitrile precursor is produced by fermentation of ethanol. In some embodiments, the fermentation is bacterial fermentation. In some embodiments, the fermentation is fungal fermentation. In some embodiments, the fermentation is anaerobic bacterial fermentation. In some embodiments, the fermentation is aerobic bacterial fermentation.

In some embodiments, the fermentation is conducted as a cyclical production process.

In some embodiments, such as shown in FIG. 5, the cyclical production process is a batch process. In some embodiments, the batch process comprises a charge phase. In some embodiments, the batch process comprises a fermentation phase. In some embodiments, the batch process comprises a discharge phase. In some embodiments, the batch process comprises a charge phase, a fermentation phase, a discharge phase, or a combination thereof.

In some embodiments, such as shown in FIG. 5, the cyclical production process is a fed-batch process. In some embodiments, the fed-batch process comprises a charge phase. In some embodiments, the fed-batch process comprises a fermentation start phase. In some embodiments, the fed-batch process comprises a substrate feed phase. In some embodiments, the fed-batch process comprises a fermentation finish phase. In some embodiments, the fed-batch process comprises a discharge phase. In some embodiments, the fed-batch process comprises a charge phase, a fermentation start phase, a substrate feed phase, a fermentation finish phase, a discharge phase, or a combination thereof.

In some embodiments, such as shown in FIG. 5, the fermentation is conducted as a continuous process. In some embodiments, the continuous process comprises a continuous inflow of mash balanced with simultaneous withdrawal of fermentation broth such that the fermentation reaches a “steady state.”

In some instances, the fermentation comprises use of a feedstock comprising ethanol. In some instances, the fermentation comprises use of a feedstock comprising sugar. The sugar may be a sugar from corn, sugar beets, sugar cane, or a combination thereof.

In some embodiments, the acetonitrile precursor (e.g., acetic acid) is impure. In some embodiments, the impure acetonitrile precursor (e.g., acetic acid) is provided in a solution comprising at most 40 wt % (e.g., at most 35 wt %, 30 wt %, 25 wt %, 20 wt %, 15 wt %, 10 wt %, or 5 wt %) of the acetonitrile precursor (e.g., acetic acid).

In some embodiments, the impurities in the impure acetonitrile precursor may comprise any organic or inorganic impurities.

In some embodiments, the acetonitrile precursor (e.g., acetic acid) is pure. In some embodiments, the pure acetonitrile precursor (e.g., acetic acid) is provided in a solution comprising at least 80 wt % (e.g., at least 85 wt %, 90 wt %, 95 wt %, 97 wt %, 98 wt %, 99 wt %, or 99.5 wt %) of the acetonitrile precursor (e.g., acetic acid).

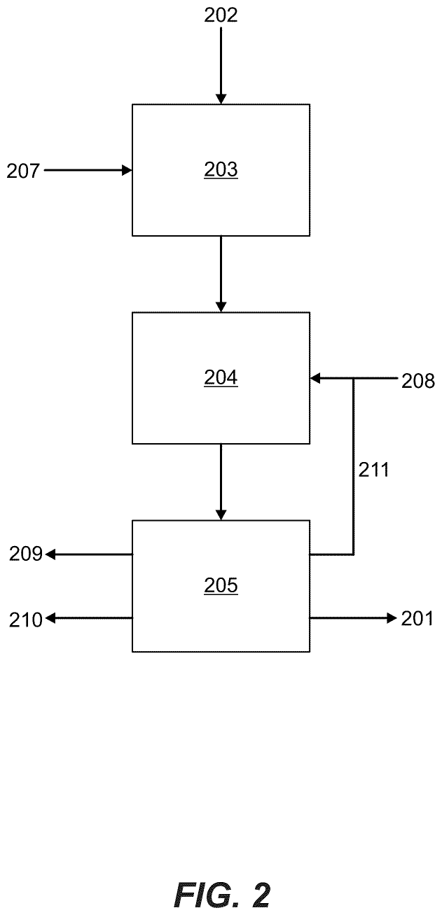

In some embodiments, the methods provided herein comprise purifying the (e.g., biologically produced) acetonitrile precursor, such as shown in FIG. 1 (103) and FIG. 2 (203).

In some embodiments, the purifying may comprise a plurality of steps.

The purifying may comprise filtration. In some embodiments, filtration removes rejected biomass. In some embodiments, filtration removes cells (e.g., from bacterial fermentation). In some embodiments, filtration comprises hollow fiber tangential flow filtration, spiral wound tangential flow filtration, flat sheet tangential flow filtration, dynamic filtration, dead-end filtration, direct flow filtration, ceramic membrane filtration, crossflow filtration, ultrafiltration, depth filtration, or nanofiltration. In some embodiments, filtration comprises spiral wound tangential flow filtration. In some embodiments, filtration comprises hollow fiber tangential flow filtration. In some embodiments, filtration comprises flat sheet tangential flow filtration. In some embodiments, filtration comprises dynamic filtration. In some embodiments, filtration comprises dead-end filtration. In some embodiments, filtration comprises dead-end filtration. In some embodiments, filtration comprises direct flow filtration. In some embodiments, filtration comprises ceramic membrane filtration. In some embodiments, filtration comprises crossflow filtration. In some embodiments, filtration comprises ultrafiltration. In some embodiments, filtration comprises nanofiltration. In some embodiments, filtration comprises depth filtration.

In some embodiments, filtration comprises use of any suitable pore size. In some embodiments, filtration comprises use of filter with pore size of at least 0.05 μm (e.g., at least 0.1 μm, 0.2 μm, 0.3 μm, 0.5 μm, 0.6 μm, 0.8 μm, 1 μm, 2 μm, 3 μm, 5 μm, 6 μm, 8 μm, 10 μm, 12 μm, 13 μm, 15 μm, 16 μm, 18 μm, or at least 20 μm). In some embodiments, filtration comprises uses of a filter with pore size of at most 50 μm (e.g., at most 45 μm, 40 μm, 35 μm, 30 μm, 25 μm, 20 μm, 15 μm, 10 μm, 8 μm, 6 μm, 5 μm, 4 μm, 2 μm, or at most 1 μm). In some embodiments, filtration comprises use of a filter with pore size of from about 0.05 μm to about 50 μm, from about 0.1 μm to about 20 μm, from about 0.5 μm to about 20 μm, from about 1 μm to about 20 μm, from about 0.1 μm to about 10 μm, from about 0.1 μm to about 1 μm, or from about 0.1 μm to about 0.8 μm. In some embodiments, filtration comprises use of a filter with pore size of about 0.1 μm, 0.2 μm, 0.3 μm, 0.4 μm, 0.5 μm, 0.6 μm, 0.7 μm, 0.8 μm, 0.9 μm, or 1 μm. In some embodiments, the pore size is 0.2 μm. In some embodiments, the pore size is 0.45 μm. In some embodiments, the pore size is 0.5 μm.

In some embodiments, filtration comprises use of spiral wound tangential flow filtration with pore size of about 0.5 μm. In some embodiments, filtration comprises use of spiral wound tangential flow filtration with pore size of about 0.2 μm. In some embodiments, filtration comprises use of spiral wound tangential flow filtration with pore size of about 0.45 μm.

In some embodiments, filtration comprises filtering the (e.g., biologically produced) acetonitrile precursor using a plurality of filters, such as filter with pore size described herein. In some embodiments, filtration comprises filtering the (e.g., biologically produced) acetonitrile precursor using 2 filters. In some embodiments, filtration comprises filtering the (e.g., biologically produced) acetonitrile precursor using 3 filters. In some embodiments, filtration comprises filtering the (e.g., biologically produced) acetonitrile precursor using 4 filters. In some embodiments, the plurality of filters (e.g., 2 or more filters) comprise differing pore sizes, such as pores sizes described elsewhere herein. For instance, one filter may comprise a pore size of about 0.5 μm, while the second filter may comprise a pore size of less than 0.5 μm. In some embodiments, the first filter may comprise a greater pore size than the second filter.

In some embodiments, filtration membrane materials comprise polyethersulfone (PES), polytetrafluoroethylene (PTFE or Teflon), polyvinylidene fluoride (PVDF), polypropylene (PP), cellulose acetate, polysulfone, nylon, polyethylene, polyvinyl alcohol (PVA), or ceramic tubular filter materials. In some embodiments, filtration membrane materials comprise polyethersulfone (PES) or polytetrafluoroethylene (PTFE or Teflon).

In some embodiments, filtration comprises use of a filter aid. In some embodiments, filtration is completed absent the use of a filter aid. In instances where a filter aid is used, a filter aid may comprise diatomaceous earth, activated charcoal, cellulose, perlite, activated alumina, bentonite, silica gel, fuller's earth, or cotton.

In some embodiments, filtration of the (e.g., biologically produced) acetonitrile precursor provides a clarified solution. In some instances, when the acetonitrile precursor is acetic acid, the clarified solution comprises acetic acid, ethanol, and water.

The clarified solution may comprise at least 5 wt % (e.g., at least 7 wt %, 8 wt %, 10 wt %, 12 wt %, 14 wt %, 16 wt %, 18 wt %, or at least 20 wt %) of the (e.g., biologically produced) acetonitrile precursor. The clarified solution may comprise at most 50 wt % (e.g., at most 45 wt %, 40 wt %, 35 wt %, 30 wt %, 28 wt %, 26 wt %, 25 wt %, 24 wt %, 22 wt %, or 20 wt %) of the (e.g., biologically produced) acetonitrile precursor.

The purifying may comprise liquid-liquid extraction, distillation, or a combination thereof. The combination of liquid-liquid extraction and distillation may be referred to as hybrid-extraction-distillation, such as described in Example 1 and Example 2.

The purifying may comprise liquid-liquid extraction. In some embodiments, the liquid-liquid extraction comprises contacting the (e.g., biologically produced) acetonitrile precursor with an organic solvent (FIG. 1 (108), FIG. 2 (207)). The organic solvent may be any suitable organic solvent in order to purify the acetonitrile precursor. In some embodiments, the organic solvent is ethyl acetate, butyl acetate, diethyl ether, dichloromethane, toluene, chloroform, methyl tert-butyl ether, toluene, chloroform, hexane, benzene, acetone, or a combination thereof. In some embodiments, the organic solvent comprises ethyl acetate. In some embodiments, the organic solvent comprises butyl acetate. In some embodiments, the organic solvent is diethyl ether. In some embodiments, the organic solvent is dichloromethane. In some embodiments, the organic solvent is toluene. In some embodiments, the organic solvent is chloroform. In some embodiments, the organic solvent is methyl tert-butyl ether. In some embodiments, the organic solvent is toluene. In some embodiments, the organic solvent is chloroform. In some embodiments, the organic solvent is hexane. In some embodiments, the organic solvent is benzene. In some embodiments, the organic solvent is acetone. In some embodiments, liquid-liquid extraction is performed once, twice, or three or more times during purification of the acetonitrile precursor. In some embodiments, liquid-liquid extraction is not performed.

In some embodiments, the extraction comprises agitation. In some embodiments, the agitation comprises vertical agitation, horizontal agitation, or a combination thereof. In some embodiments, the agitation comprises vertical (axial) agitation. In some embodiments, the agitation comprises horizontal agitation. In some embodiments, the agitation does not comprise horizontal agitation. In some embodiments, the agitation does not comprise vertical agitation.

In some embodiments, purifying comprises solid phase extraction (SPE). In some embodiments, the SPE is normal phase SPE, reverse phase SPE, or mixed-mode SPE. In some embodiments, the adsorbent used in SPE is silica gel, alumina, C18, C8, C2, Oasis HLB, or Strata X. In some embodiments, the adsorbent used in SPE is silica gel or alumina. In some embodiments, the adsorbent used in C18, C8, or C2. In some embodiments, the adsorbent used in Oasis HLB or Strata X.

The purifying may comprise distillation. Distillation may be used to remove one or more organic solvents present in the (e.g., biologically produced) acetonitrile precursor. The one or more organic solvents may comprise ethanol (e.g., from the fermentation process described elsewhere herein) or an organic solvent described hereinabove. In some embodiments, distillation is performed once, twice, or three or more times during purification of the acetonitrile precursor. In some embodiments, distillation is not performed.

The purifying may comprise stripping. Stripping may be used to remove one or more organic solvents present in the (e.g., biologically produced) acetonitrile precursor. The one or more organic solvents may comprise ethanol (e.g., from the fermentation process described elsewhere herein) or an organic solvent described hereinabove. In some embodiments, stripping is performed once, twice, or three or more times during purification of the acetonitrile precursor. In some embodiments, stripping is not performed.

In some embodiments, excess water may be removed from the (e.g., biologically produced) acetonitrile precursor.

In some embodiments, residual water and organic solvent removed from the acetonitrile precursor during purification is recycled. In some embodiments, water, and ethanol (e.g., from the fermentation described hereinabove) is recycled and used in further fermentation. In some embodiments, recycling of water and/or ethanol is shown in FIG. 1 (107). In some embodiments, recycling of water and/or ethanol (shown in FIG. 1 (107)) is optional. In some cases, the water and/or ethanol is removed as a waste stream (FIG. 1 (113)).

In some embodiments, the product of purifying the (e.g., biologically produced) acetonitrile precursor is a purified (e.g., biologically produced) acetonitrile precursor. In some embodiments, the (e.g., biologically produced) acetonitrile precursor is at least 85% (e.g., at least 87%, 88%, 90%, 92%, 94%, 95%, 96%, 97%, 98%, 99%, 99.5%, 99.7%, 99.9%, or 99.99%) pure. In some embodiments, the (e.g., biologically produced) acetonitrile precursor is from about 85% to about 99.99% pure, from about 90% to about 99.9% pure, from about 90% to about 99.5% pure, from about 95% to about 99.9% pure, or from about 95% to about 99% pure. In some embodiments, the purified acetonitrile precursor is anhydrous. In some instances, the anhydrous purified acetonitrile precursor comprises no more than 30 ppm of water.

In some embodiments, the (e.g., biologically produced) acetonitrile precursor comprises no more than 15 wt % (e.g., no more than 13 wt %, 12 wt %, 10 wt %, 8 wt %, 6 wt %, 5 wt %, 4 wt %, 3 wt %, 2 wt %, 1 wt %, 0.5 wt %, 0.3 wt %, 0.1 wt %, or 0.01 wt %) of impurities, such as after purification. In some embodiments, the (e.g., biologically produced) acetonitrile precursor comprises no more than 0.01 wt % to 15 wt % of impurities, from about 0.1 wt % to 10 wt % of impurities from 0.5 wt % to 10 wt % of impurities from 0.1 wt % to 5 wt % of impurities, or from 1 wt % to 5 wt % of impurities.

In specific embodiments, the (e.g., biologically produced) acetonitrile precursor is acetic acid. In some embodiments, purifying the acetonitrile precursor provides glacial acetic acid. In some embodiments, the acetic acid is at least 85% (e.g., at least 87%, 88%, 90%, 92%, 94%, 95%, 96%, 97%, 98%, 99%, 99.5%, 99.7%, 99.9%, or 99.99%) pure. In some embodiments, the acetic acid is from about 85% to about 99.99% pure, from about 90% to about 99.9% pure, from about 90% to about 99.5% pure, from about 95% to about 99.9% pure, or from about 95% to about 99% pure. In some embodiments, the purified acetic acid (e.g., glacial acetic acid) is anhydrous.

In some embodiments, impurities in acetic acid comprise any organic or inorganic impurities other than acetic acid.

In some embodiments, the (e.g., purified) acetic acid comprises no more than 15 wt % (e.g., no more than 13 wt %, 12 wt %, 10 wt %, 8 wt %, 6 wt %, 5 wt %, 4 wt %, 3 wt %, 2 wt %, 1 wt %, 0.5 wt %, 0.3 wt %, 0.1 wt %, or 0.01 wt %) of impurities, such as after purification. In some embodiments, the (e.g., purified) acetic acid comprises no more than 0.01 wt % to 15 wt % of impurities, from about 0.1 wt % to 10 wt % of impurities, from 0.5 wt % to 10 wt % of impurities, from 0.1 wt % to 5 wt % of impurities, or from 1 wt % to 5 wt % of impurities.

In some embodiments, the methods provided herein comprise reacting the (e.g., biologically produced) acetonitrile precursor with a nitrogen source to provide (e.g., crude) acetonitrile.

The reaction of the acetonitrile precursor with the nitrogen source to provide acetonitrile may be performed using any suitable method. In some embodiments, the reaction is called nitrilation.

In some embodiments, nitrilation includes a combination of (1) ammonolysis to produce an amide and (2) amide dehydration.

In some embodiments, the reacting of the acetonitrile precursor with the nitrogen source does not produce hydrogen cyanide.

In some embodiments, the reacting of the acetonitrile precursor with the nitrogen source does not produce acrylonitrile.

In some embodiments, the nitrogen source is ammonia, an ammonium salt (e.g., ammonium bicarbonate, ammonium carbonate, ammonium carbamate, ammonium chloride, ammonium acetate, or ammonium sulfate), or ammonium hydroxide. In some embodiments, the nitrogen source is ammonia. In some embodiments, the nitrogen source is an ammonium salt (e.g., ammonium bicarbonate, ammonium carbonate, ammonium carbamate, ammonium chloride, ammonium acetate, or ammonium sulfate). In some embodiments, the nitrogen source is ammonium hydroxide. In some embodiments, the ammonia is provided as a recycled (unreacted) product of subsequent steps (e.g., as shown in FIG. 1 (112), FIG. 2 (211)). In some embodiments, the recycling of ammonia from subsequent steps (e.g., as shown in FIG. 1 (112), FIG. 2 (211)) is optional.

In some embodiments, the reaction is completed at elevated temperature. In some embodiments, the reaction is completed at a temperature of at least 200° C. (e.g., at least 210° C., 220° C., 230° C., 240° C., 250° C., 260° C., 270° C., 280° C., 290° C., or at least 300° C.). In some embodiments, the reaction is completed at a temperature of no more than 350° C. (e.g., no more than 340° C., 330° C., 320° C., 310° C., or 300° C.). In some embodiments, the reaction is completed at a temperature of no more than 450° C. (e.g., no more than 425° C., 400° C., 380° C., 370° C., or 360° C.). In some embodiments, the reaction is completed at a temperature of from about 200° C. to about 450° C., from about 200° C. to about 400° C., from about 200° C. to about 350° C., from about 220° C. to about 250° C., from about 250° C. to about 330° C., from about 290° C. to about 340° C., or from about 290° C. to about 320° C. In some embodiments, the reaction is completed at a temperature of about 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 305° C., 310° C., 315° C., 320° C., 325° C., or about 330° C.

In some embodiments, reactants are pre-heated before reaction, such as to prevent formation of unfavorable side products. In some embodiments, pre-heating comprises heating to a temperature of at least 180° C. (e.g., at least 190° C., 200° C., 210° C., 220° C., 230° C., 240° C., 250° C., or at least 260° C.).

As described herein in Example 1 and Example 2, side reactions may become more pronounced at higher temperatures and reactor fouling may occur at lower temperatures due to incomplete reaction.

In some embodiments, the reaction is completed at standard pressure, below standard pressure, or at elevated pressures. In some embodiments, the reaction is completed at a combination of pressures at, above, or below standard pressure. In some embodiments, the reaction is completed at standard pressure. In some embodiments, the reaction is completed below standard pressure. In some embodiments, the reaction is completed at elevated pressure. In some embodiments, the reaction is completed at an absolute pressure of at least 0.4 atm (e.g., at least 0.45 atm, 0.4 atm, 0.55 atm, 0.6 atm, 0.55 atm, 0.65 atm, 0.7 atm, 0.75 atm, 0.8 atm, 0.85 atm, 0.9 atm, 0.95 atm, 0.1 atm, 0.2 atm, 0.4 atm, 0.5 atm, 1 atm, 1.25 atm, 1.5 atm, 1.75 atm, 2 atm, 3 atm, 4 atm, 5 atm, 6 atm, 7 atm, 8 atm, 9 atm, or at least 10 atm). In some embodiments, the reaction is completed at a pressure of at most 10 atm (e.g., at most 9 atm, 8 atm, 7 atm, 6 atm, 5 atm, 4 atm, 3 atm, 2 atm, or 1 atm). In some embodiments, the reaction is completed at a pressure of from about 1 atm to about 10 atm, from about 1 atm to about 8 atm, from about 1 atm to about 6 atm, from about 1 atm to about 5 atm, from about 1 atm to about 3 atm, or from about 1 atm to about 4 atm. In some embodiments, the reaction is completed at a temperature of about 1 atm, 1.2 atm, 1.4 atm, 1.6 atm, 1.8 atm, 2 atm, 2.2 atm, 2.4 atm, 2.6 atm, 2.8 atm, 3 atm, 3.2 atm, 3.4 atm, 3.6 atm, 3.8 atm, 4 atm, 4.2 atm, 4.4 atm, 4.6 atm, 4.8 atm, or about 5 atm. In some instances, the pressure is absolute pressure.

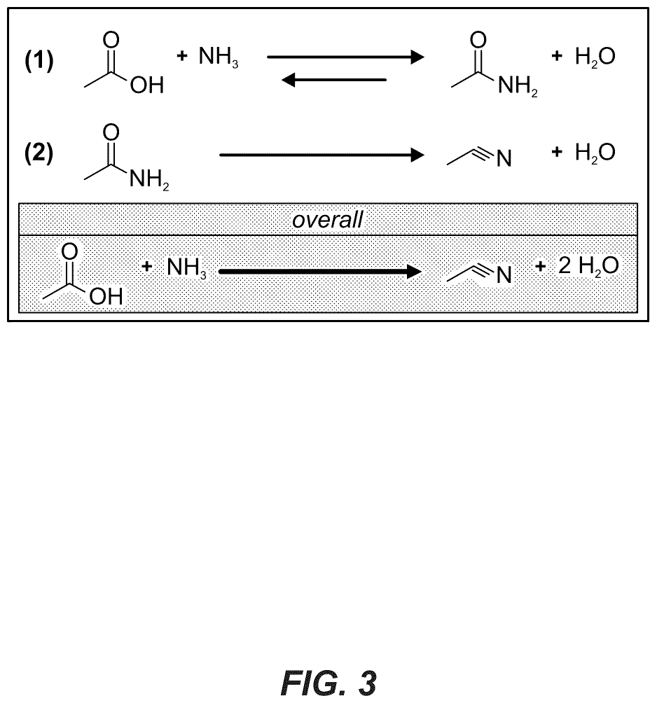

In some embodiments, the reaction comprises contacting (e.g., glacial) acetic acid as provided or produced in the preceding steps with a nitrogen source (e.g., ammonia) to produce (e.g., crude) acetonitrile, as depicted in FIG. 3.

In some embodiments, the reaction comprises use of a catalyst. In some embodiments, the catalyst is a heterogeneous catalyst. In other embodiments, the catalyst is a homogeneous catalyst. In some embodiments, the catalyst is a metal oxide. In some embodiments, the catalyst is a transition metal oxide. In some embodiments, the catalyst is aluminum oxide, titanium oxide, zirconium oxide, tungsten oxide, or a combination thereof. In some embodiments, the catalyst is aluminum oxide. In some embodiments, the catalyst is titanium oxide. In some embodiments, the catalyst is zirconium oxide. In some embodiments, the catalyst is tungsten oxide.

The catalyst may be contained in a fixed bed reactor, a trickle-bed reactor, a moving bed reactor, a rotating bed reactor, a slurry reactor, or a fluidized bed reactor.

In some embodiments, the reacting provided herein produces no more than 30 wt % of organic products other than acetonitrile. In some embodiments, the reacting provided herein produces no more than 20 wt % of organic products other than acetonitrile. In some embodiments, the reacting provided herein produces no more than 15 wt % of organic products other than acetonitrile. In some embodiments, the reacting provided herein produces no more than 10 wt % of organic products other than acetonitrile. In some embodiments, the reacting provided herein produces no more than 9 wt % of organic products other than acetonitrile. In some embodiments, the reacting provided herein produces no more than 8 wt % of organic products other than acetonitrile. In some embodiments, the reacting provided herein produces no more than 7 wt % of organic products other than acetonitrile. In some embodiments, the reacting provided herein produces no more than 6 wt % of organic products other than acetonitrile. In some embodiments, the reacting provided herein produces no more than 5 wt % of organic products other than acetonitrile.

In some embodiments, the methods herein comprise purifying the (e.g., crude) acetonitrile (e.g., as depicted in FIG. 1 (105), FIG. 2 (205)).

In some embodiments, the methods provided herein comprise purifying (e.g., crude) acetonitrile that is produced by any suitable method, such as propylene ammoxidation.

In some embodiments, purifying the (e.g., crude) acetonitrile comprises distillation, oxidation, polishing, pervaporation, hybrid extraction-distillation, extractive distillation, salting out, sugaring out, freezing out (e.g., crystallization), or a combination thereof. In some embodiments, purifying the (e.g., crude) acetonitrile comprises distillation. In some embodiments, purifying the (e.g., crude) acetonitrile comprises oxidation. In some embodiments, purifying the (e.g., crude) acetonitrile comprises polishing. In some embodiments, purifying the (e.g., crude) acetonitrile comprises pervaporation. In some embodiments, purifying the (e.g., crude) acetonitrile comprises hybrid extraction-distillation. In some embodiments, purifying the (e.g., crude) acetonitrile comprises extractive distillation. In some embodiments, purifying the (e.g., crude) acetonitrile comprises salting out. In some embodiments, purifying the (e.g., crude) acetonitrile comprises sugaring out. In some embodiments, purifying the (e.g., crude) acetonitrile comprises freezing out (e.g., crystallization).

In some embodiments, purifying the (e.g., crude) acetonitrile comprises distillation. In some embodiments, the distillation removes unreacted nitrogen source from the (e.g., crude) acetonitrile. In some embodiments, the distillation removes at least 85 wt % (e.g., at least 90 wt %, 92 wt %, 94 wt %, 95 wt %, 96 wt %, 97 wt %, 98 wt %, 99 wt %, 99.5 wt %, 99.9 wt %, or at least 99.99 wt %) of the unreacted nitrogen source from the (e.g., crude) acetonitrile. In some embodiments, the distillation removes from about 85 wt % to about 100 wt %, from about 90 wt % to about 100 wt %, from about 94 wt % to about 100 wt %, or from about 97 wt % to about 100 wt % of the unreacted nitrogen source from the (e.g., crude) acetonitrile.

In some embodiments, the nitrogen source removed from the (e.g., crude) acetonitrile may be recycled. The recycled nitrogen source may be used in the reaction step with the acetonitrile precursor, as described elsewhere herein (FIG. 1 (112), FIG. 2 (211)). In some embodiments, the nitrogen source removed from the (e.g., crude) acetonitrile may be removed and stored. The nitrogen source may be removed and stored by use of e.g., cryogenic distillation or an entrainer.

In some embodiments, purifying the (e.g., crude) acetonitrile comprises distillation. In some embodiments, the distillation removes unreacted nitrogen sources from the (e.g., crude) acetonitrile. In some embodiments, the distillation removes at least 85 wt % (e.g., at least 90 wt %, 92 wt %, 94 wt %, 95 wt %, 96 wt %, 97 wt %, 98 wt %, 99 wt %, 99.5 wt %, 99.9 wt %, or at least 99.99 wt %) of the nitrogen source from the (e.g., crude) acetonitrile. In some embodiments, the distillation removes from about 85 wt % to about 100 wt %, from about 90 wt % to about 100 wt %, from about 94 wt % to about 100 wt %, or from about 97 wt % to about 100 wt % of the nitrogen source from the (e.g., crude) acetonitrile.

In some embodiments, the nitrogen source removed from the (e.g., crude) acetonitrile may be recycled. The recycled nitrogen source may be used in the reaction step with the acetonitrile precursor, as described elsewhere herein (FIG. 1 (112), FIG. 2 (211)). In some embodiments, the nitrogen source removed from the (e.g., crude) acetonitrile may be removed and stored. The nitrogen source may be removed and stored by use of e.g., cryogenic distillation or an entrainer.

In some embodiments, distillation removes volatile impurities from the (e.g., crude) acetonitrile. The volatile impurities may include ammonia, carbon dioxide, acetone, or a combination thereof. In some embodiments, distillation removes carbon dioxide from the (e.g., crude) acetonitrile. In some embodiments, distillation removes acetone from the (e.g., crude) acetonitrile (FIG. 1 (111), FIG. 2 (210)).

In some instances, impurities present in the (e.g., crude) acetonitrile include acetone and carbon dioxide. These impurities may be a result of ketonization of acetic acid.

In some embodiments, distillation comprises pressure swing distillation, extractive distillation, azeotropic distillation, heterogeneous azeotropic distillation, or vacuum distillation. In some embodiments, the distillation comprises pressure swing distillation. In some embodiments, the distillation comprises extractive distillation. In some embodiments, the distillation comprises azeotropic distillation. In some embodiments, the distillation comprises heterogeneous azeotropic distillation. In some embodiments, the distillation comprises vacuum distillation.

In some embodiments, impurities in the (e.g., crude) acetonitrile include other nitriles (e.g., 3-methyl 2-butene nitrile and propionitrile), aromatics, acetone, carbon dioxide, ammonium salts, water, or a combination thereof. In some instances, the unsaturated nitriles may be present at a concentration of about 100 ppm. In some instances, the total aromatics may be present at a concentration of about 20 ppm. Further purification may be required to meet certain specifications, such as for oligonucleotide grade quality, as described elsewhere herein.

In some embodiments, purifying the (e.g., crude) acetonitrile comprises oxidation, e.g., oxidation of impurities. In some cases, the oxidation of impurities allows for more facile removal of the impurities from the (e.g., crude) acetonitrile enabling further purification.

In some embodiments, purifying the (e.g., crude) acetonitrile comprises use of an oxidant. In some embodiments, the oxidant is a Lewis acid. In some embodiments, the oxidant is potassium permanganate, ozone, potassium superoxide, potassium persulfate, hydrogen peroxide, sodium hypochlorite, sodium persulfate, nitric acid, sulfuric acid, chlorine dioxide, or lead dioxide. In some embodiments, the oxidant is potassium permanganate, ozone, or potassium superoxide. In some embodiments, the oxidant is potassium permanganate. In some embodiments, the oxidant is ozone. In some embodiments, the oxidant is potassium superoxide. In some embodiments, the oxidant can include hydrogen peroxide. In some embodiments, the oxidation can involve UV light.

In some embodiments, purifying the (e.g., crude) acetonitrile comprises use of acidic, basic, or neutral conditions. In some embodiments, purifying the (e.g., crude) acetonitrile comprises use of acidic conditions. In some embodiments, purifying the (e.g., crude) acetonitrile comprises use of neutral conditions. In some embodiments, purifying the (e.g., crude) acetonitrile comprises use of basic conditions.

In some embodiments, the oxidation is completed in acidic, basic, or neutral conditions. In some embodiments, the oxidation is completed in acidic conditions. Acidic conditions may be provided by addition of hydrochloric acid, hydrobromic acid, sulfuric acid, nitric acid, perchloric acid, or any other suitable acid. In some embodiments, the oxidation is completed in neutral conditions. In some embodiments, neutral conditions comprise use of no acid or base. In some embodiments, neutral conditions comprise use of a buffer. In some embodiments, the oxidation is completed in basic conditions. Basic conditions may be provided by addition of a base, such as sodium hydroxide, potassium hydroxide, calcium hydroxide, magnesium hydroxide, or lithium hydroxide.

In some embodiments, the purifying comprises polishing. In some embodiments, purifying the (e.g., crude) acetonitrile (polishing) comprises contacting the (e.g., crude) acetonitrile with a zeolite, ion-exchange resin, activated carbon, silica, calcium sulfate, molecular sieves, alumina, activated alumina, magnesium sulfate, or a combination thereof. In some embodiments, purifying the (e.g., crude) acetonitrile (polishing) comprises contacting the (e.g., crude) acetonitrile with at least two of a zeolite, ion-exchange resin, activated carbon, silica, calcium sulfate, molecular sieves, alumina, activated alumina, and magnesium sulfate.

In some embodiments, purifying the (e.g., crude) acetonitrile (polishing) comprises first contacting the (e.g., crude) acetonitrile with a first polishing material followed by contacting the (e.g., crude) acetonitrile with a second polishing material. In some embodiments, purifying the (e.g., crude) acetonitrile (polishing) comprises first contacting the (e.g., crude) acetonitrile with a first polishing material. In some embodiments, purifying the (e.g., crude) acetonitrile (polishing) comprises first contacting the (e.g., crude) acetonitrile with a second polishing material. In some embodiments, the polishing material is a zeolite, ion-exchange resin, activated carbon, silica, calcium sulfate, molecular sieves, alumina, activated alumina, magnesium sulfate, or a combination thereof. In some embodiments, the first polishing material is a zeolite, ion-exchange resin, activated carbon, silica, calcium sulfate, molecular sieves, alumina, activated alumina, or magnesium sulfate. In some embodiments, the second polishing material is a zeolite, ion-exchange resin, activated carbon, silica, calcium sulfate, molecular sieves, alumina, activated alumina, or magnesium sulfate. In some embodiments, the first polishing material and the second polishing material are the same. In some embodiments, the first polishing material and the second polishing material are different.

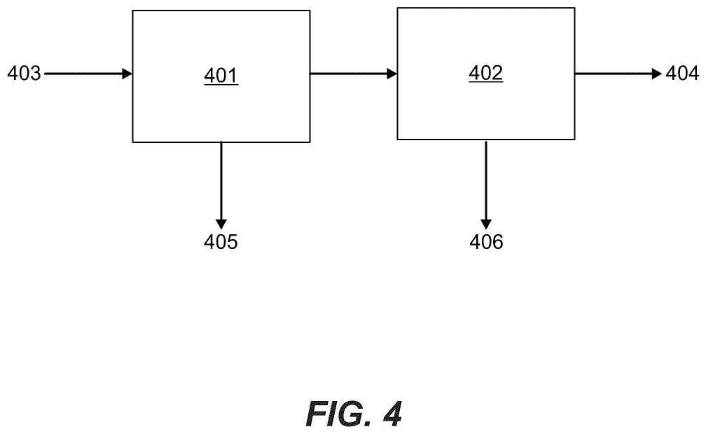

In some embodiments, purifying the (e.g., crude) acetonitrile (polishing) comprises first contacting the (e.g., crude) acetonitrile with a zeolite, ion-exchange resin, activated carbon, silica, calcium sulfate, molecular sieves, alumina, activated alumina, or magnesium sulfate (FIG. 4 (401)), followed by second, contacting the (e.g., crude) acetonitrile (polishing) with a (e.g., second portion of a) zeolite, ion-exchange resin, activated carbon, silica, calcium sulfate, molecular sieves, alumina, activated alumina, and magnesium sulfate (FIG. 4 (402)). In some embodiments, purifying the (e.g., crude) acetonitrile (polishing) comprises first contacting the (e.g., crude) acetonitrile with a first zeolite, ion-exchange resin, activated carbon, silica, calcium sulfate, molecular sieves, alumina, activated alumina, magnesium sulfate, or a combination thereof (FIG. 4 (401)). In some embodiments, purifying the (e.g., crude) acetonitrile (polishing) comprises contacting the (e.g., crude) acetonitrile with a second zeolite, ion-exchange resin, activated carbon, silica, calcium sulfate, molecular sieves, alumina, activated alumina, magnesium sulfate, or a combination thereof (FIG. 4 (402)). In some embodiments, the zeolite, ion-exchange resin, activated carbon, silica, calcium sulfate, molecular sieves, alumina, activated alumina, magnesium sulfate, or a combination thereof in 401 is the same as the zeolite, ion-exchange resin, activated carbon, silica, calcium sulfate, molecular sieves, alumina, activated alumina, magnesium sulfate, or a combination thereof in 402. In some embodiments, the zeolite, ion-exchange resin, activated carbon, silica, calcium sulfate, molecular sieves, alumina, activated alumina, magnesium sulfate, or a combination thereof in 401 is different than the zeolite, ion-exchange resin, activated carbon, silica, calcium sulfate, molecular sieves, alumina, activated alumina, magnesium sulfate, or a combination thereof in 402.

In some embodiments, purifying the (e.g., crude) acetonitrile (polishing) comprises contacting the (e.g., crude) acetonitrile with a zeolite. In some embodiments, purifying the (e.g., crude) acetonitrile comprises contacting the (e.g., crude) acetonitrile (polishing) with an ion-exchange resin. In some embodiments, purifying the (e.g., crude) acetonitrile (polishing) comprises contacting the (e.g., crude) acetonitrile with activated carbon. In some embodiments, purifying the (e.g., crude) acetonitrile (polishing) comprises contacting the (e.g., crude) acetonitrile with silica. In some embodiments, purifying the (e.g., crude) acetonitrile (polishing) comprises contacting the (e.g., crude) acetonitrile with calcium sulfate. In some embodiments, purifying (polishing) the (e.g., crude) acetonitrile comprises contacting (polishing) the (e.g., crude) acetonitrile with molecular sieves. In some embodiments, purifying the (e.g., crude) acetonitrile (polishing) comprises contacting the (e.g., crude) acetonitrile with alumina. In some embodiments, purifying the (e.g., crude) acetonitrile (polishing) comprises contacting the (e.g., crude) acetonitrile with activated alumina. In some embodiments, purifying the (e.g., crude) acetonitrile (polishing) comprises contacting the (e.g., crude) acetonitrile with magnesium sulfate.

In some embodiments, purifying the (e.g., crude) acetonitrile (polishing) comprises contacting the (e.g., crude) acetonitrile (403) with activated carbon (401), such as to remove impurity aromatic compounds (405) such as depicted in FIG. 4. In some embodiments, purifying the (e.g., crude) acetonitrile (polishing) comprises contacting the (e.g., crude) acetonitrile with activated alumina (402), such as to remove trace water (406) such as depicted in FIG. 4. The contacting with e.g., alumina or activated carbon (or any other purification reagents described herein) may be completed in packed absorption columns.

In some embodiments, the scheme shown in FIG. 4 is comprised in FIG. 1 (105) or FIG. 2 (205).

In some embodiments the resulting acetonitrile after purification of the (e.g., crude) acetonitrile is purified acetonitrile (FIG. 1 (101), FIG. 2 (201)).

The acetonitrile prepared by the methods provided herein may be prepared at both lab- and industrial-scales. In some embodiments, the method produces acetonitrile (compositions) in a quantity of at least 100 mL (e.g., at least 500 mL, 1 L, 5 L, 10 L, 15 L, 20 L, or 25 L). In other embodiments, the method produces acetonitrile (compositions) in a quantity of at least 100 L (e.g., at least 150 L, 200 L, 250 L, 300 L, 350 L, 400 L, 450 L, 500 L, 600 L, 700 L, 800 L, 900 L, 1000 L, 2500 L, 5000 L, 7500 L, or 10000 L).

In some embodiments, the methods provided herein may prepare acetonitrile (compositions) at a rate of at least 0.5 kg/h (e.g., at least 0.75 kg/h, 1 kg/h, 1.5 kg/h, 2 kg/h, 2.5 kg/h, 3 kg/h, 4 kg/h, 5 kg/h, 6 kg/h, 7 kg/h, 8 kg/h, 9 kg/h, 10 kg/h, or more). In some embodiments, the methods herein prepare acetonitrile (compositions) at a rate of at least 5 kg/h. In some embodiments, the methods herein prepare acetonitrile at a rate of at least 6 kg/h.

### Purified Bio-Acetonitrile

In some embodiments, provided herein are acetonitrile compositions prepared by any of the methods provided herein. In some embodiments, the acetonitrile compositions provided herein, prepared by the methods provided herein, may be, for example, oligonucleotide synthesis grade, ACS grade, HPLC grade, or LC/MS grade acetonitrile compositions.

In some embodiments, the acetonitrile composition is at least 90% pure. In some embodiments, the acetonitrile composition is at least 95% pure. In some embodiments, the acetonitrile composition is at least 97.5% pure. In some embodiments, the acetonitrile composition is at least 99% (e.g., at least 99.1%, 99.2%, 99.3%, 99.4%, 99.5%, 99.6%, 99.7%, 99.8%, or 99.9%) pure. In some embodiments, the acetonitrile composition is at least 99.6% pure. In some embodiments, the acetonitrile composition is at least 99.7% pure. In some embodiments, the acetonitrile composition is at least 99.8% pure. In some embodiments, the acetonitrile composition is at least 99.9% pure. In some embodiments, the acetonitrile composition is from about 90% to about 99.99% pure, about 95% to about 99.99% pure, about 97% to about 99.99% pure, about 98% to about 99.99% pure, or about 99% to about 99.99% pure.

The purity of the acetonitrile compositions described herein may be measured by gas chromatography (GC) coupled with mass spectrometry (MS), flame ionization detectors (FID), thermal conductivity detectors (TCD), or a combination thereof. In some instances, the purity of the acetonitrile composition is measured by GC-MS. In some instances, the purity of the acetonitrile composition is measured by GC-MS/FID/TCD. Absorbance of the acetonitrile compositions as described herein may be measured using UV-Vis spectroscopy, such as using a path length of 1 cm. The acetonitrile compositions provided herein (e.g., the identity of the acetonitrile) may be measured by use of Fourier Transform Infrared Spectroscopy (FTIR) or nuclear magnetic resonance (NMR) spectroscopy. The water content of the acetonitrile compositions provided herein may be measured using Karl Fischer titration.

In some embodiments, the acetonitrile composition comprises less than 10 wt % of impurities (e.g., organic, or inorganic impurities or water). In some embodiments, the acetonitrile composition comprises less than 5 wt % of impurities. In some embodiments, the acetonitrile composition comprises less than 1 wt % (e.g., less than 0.9 wt %, 0.8 wt %, 0.7 wt %, 0.6 wt %, 0.5 wt %, 0.4 wt %, 0.3 wt %, 0.2 wt %, or 0.1 wt %) of impurities. In some embodiments, the acetonitrile composition comprises no more than 0.5 wt % of impurities. In some embodiments, the acetonitrile composition comprises no more than 0.4 wt % of impurities. In some embodiments, the acetonitrile composition comprises no more than 0.3 wt % of impurities. In some embodiments, the acetonitrile composition comprises no more than 0.2 wt % of impurities. In some embodiments, the acetonitrile composition comprises no more than 0.1 wt % of impurities. In some embodiments, the acetonitrile composition comprises from 0 wt % to about 10 wt % of impurities, from about 0.01 wt % to about 5 wt % of impurities, from about 0.01 wt % to about 3 wt % of impurities, from about 0.01 wt % to about 2 wt % of impurities, or from 0.01 wt % to about 1 wt % of impurities. In some embodiments, the acetonitrile composition comprises 0.01 wt %, 0.02 wt %, 0.05 wt %, 0.1 wt %, 0.15 wt %, 0.2 wt %, 0.25 wt %, 0.3 wt %, 0.35 wt %, 0.4 wt %, 0.45 wt %, 0.5 wt %, 0.75 wt %, 1 wt %, 1.5 wt %, 2 wt %, 2.5 wt %, 3 wt % 3.5 wt %, 4 wt %, 4.5 wt %, or 5 wt % of impurities.

The amount (e.g., wt %) of impurities in the acetonitrile composition may be measured by gas chromatography.

In some embodiments, the acetonitrile impurities may comprise any organic or inorganic impurities (e.g., organic, or inorganic impurities produced by the methods provided herein).

The acetonitrile identity may be confirmed by infrared spectroscopy or nuclear magnetic resonance (NMR) spectroscopy.

In some embodiments, the density of the acetonitrile composition at 20° C. may be about 0.774 g/mL. In some embodiments, the density of the acetonitrile composition at 20° C. may be about 0.775 g/mL. In some embodiments, the density of the acetonitrile composition at 20° C. may be about 0.776 g/mL. In some embodiments, the density of the acetonitrile composition at 20° C. may be about 0.778 g/mL. In some embodiments, the density of the acetonitrile composition at 20° C. may be about 0.779 g/mL. In some embodiments, the density of the acetonitrile composition at 20° C. may be about 0.780 g/mL. In some embodiments, the density of the acetonitrile composition at 20° C. may be about 0.781 g/mL. In some embodiments, the density of the acetonitrile composition at 20° C. may be about 0.782 g/mL. In some embodiments, the density of the acetonitrile composition at 20° C. may be about 0.783 g/mL. In some embodiments, the density of the acetonitrile composition at 20° C. may be about 0.784 g/mL. In some embodiments, the density of the acetonitrile composition at 20° C. may be about 0.785 g/mL. In some embodiments, the density of the acetonitrile composition at 20° C. may be about 0.786 g/mL. In some embodiments, the density of the acetonitrile composition at 20° C. may be at least 0.774 g/mL (e.g., at least 0.776 g/mL, 0.777 g/mL, 0.778 g/mL, or 0.780 g/mL). In some embodiments, the density of the acetonitrile composition at 20° C. may be at most 0.785 g/mL (e.g., at most 0.784 g/mL, 0.783 g/mL, 0.782 g/mL, 0.781 g/mL, or 0.780 g/mL). In some embodiments, the density of the acetonitrile composition at 20° C. may be from about 0.778 g/mL to about 0.786 g/mL. In some embodiments, the density of the acetonitrile composition at 20° C. may be from about 0.780 g/mL to about 0.783 g/mL.

In some embodiments, the refractive index at 20° C. of the acetonitrile composition is about 1.340. In some embodiments, the refractive index at 20° C. of the acetonitrile composition is about 1.341. In some embodiments, the refractive index at 20° C. of the acetonitrile composition is about 1.342. In some embodiments, the refractive index at 20° C. of the acetonitrile composition is about 1.343. In some embodiments, the refractive index at 20° C. of the acetonitrile composition is about 1.344. In some embodiments, the refractive index at 20° C. of the acetonitrile composition is about 1.345. In some embodiments, the refractive index at 20° C. of the acetonitrile composition is about 1.346. In some embodiments, the refractive index at 20° C. of the acetonitrile composition is about 1.347. In some embodiments, the refractive index at 20° C. of the acetonitrile composition is about 1.348. In some embodiments, the refractive index at 20° C. of the acetonitrile composition is about 1.349. In some embodiments, the refractive index at 20° C. of the acetonitrile composition is no more than 1.349. In some embodiments, the refractive index at 20° C. of the acetonitrile composition is no more than 1.346. In some embodiments, the refractive index at 20° C. of the acetonitrile composition is from about 1.341 to about 1.349. In some embodiments, the refractive index at 20° C. of the acetonitrile composition is from about 1.343 to about 1.346.

In some embodiments, the acetonitrile composition comprises less than 1000 ppm (e.g., less than 900 ppm, 800 ppm, 700 ppm, 600 ppm, 500 ppm, 400 ppm, 300 ppm, 200 ppm, or less than 100 ppm) of water. In some embodiments, the acetonitrile composition comprises less than 100 ppm (e.g., less than 90 ppm, 80 ppm, 70 ppm, 60 ppm, 50 ppm, 40 ppm, 30 ppm, 20 ppm, 10 ppm, or less than 5 ppm) of water. In some embodiments, the acetonitrile composition comprises less than 50 ppm of water. In some embodiments, the acetonitrile composition comprises less than 20 ppm of water. In some embodiments, the acetonitrile composition comprises less than 10 ppm of water. In some embodiments, the acetonitrile composition comprises less than 5 ppm of water. In some embodiments, the acetonitrile composition comprises less than 1 ppm of water. In some embodiments, the acetonitrile composition comprises from about 1 ppm to about 50 ppm of water, from about 1 ppm to about 25 ppm of water, from about 1 ppm to about 20 ppm of water, from about 1 ppm to about 10 ppm of water, or from about 1 ppm to about 5 ppm of water.

Water content in the acetonitrile composition may be measured by Karl Fischer titration.

In some embodiments, the acetonitrile composition is anhydrous.

In some embodiments, the acetonitrile composition comprises an acidity (as CH₃COOH) of less than 0.01% (e.g., less than 0.009%, 0.008%, 0.007%, 0.006%, 0.005%, 0.004%, 0.003%, 0.002%, or 0.001%). In some embodiments, the acetonitrile composition comprises an acidity (as CH₃COOH) of less than 0.001% (e.g., less than 0.0009%, 0.0008%, 0.0007%, 0.0006%, 0.0005%, 0.0004%, 0.0003%, 0.0002%, or 0.0001%). In some embodiments, the acetonitrile composition comprises an acidity (as CH₃COOH) of less than 0.001%. In some embodiments, the acetonitrile composition comprises an acidity (as CH₃COOH) of less than 0.0005%. In some embodiments, the acetonitrile composition comprises an acidity (as CH₃COOH) of from about 0.0001% to about 0.05%, from about 0.0005% to about 0.005%, or from about 0.0008% to about 0.002%.

In some embodiments, the acetonitrile composition comprises less than 20 μeq/g (e.g., less than 18 μeq/g, 16 μeq/g, 15 μeq/g, 14 μeq/g, 13 μeq/g, 12 μeq/g, 11 μeq/g, 10 μeq/g, 9 μeq/g, 8 μeq/g, 7 μeq/g, 6 μeq/g, 5 μeq/g, 4 μeq/g, 3 μeq/g, 2 μeq/g, 1 μeq/g, 0.5 μeq/g, or less than 0.1 μeq/g) of titratable acid. In some embodiments, the acetonitrile composition comprises less than 15 μeq/g of titratable acid. In some embodiments, the acetonitrile composition comprises less than 10 μeq/g of titratable acid. In some embodiments, the acetonitrile composition comprises less than 8 μeq/g of titratable acid. In some embodiments, the acetonitrile composition comprises less than 5 μeq/g of titratable acid. In some embodiments, the acetonitrile composition comprises from about 0.1 μeq/g to about 20 μeq/g, about 0.1 μeq/g to about 15 μeq/g, about 0.1 μeq/g to about 10 μeq/g, or from about 0.1 μeq/g to about 5 μeq/g of titratable acid.

In some embodiments, the acetonitrile composition comprises less than 10 μeq/g (e.g., less than 8 μeq/g, 6 μeq/g, 5 μeq/g, 4 μeq/g, 3 μeq/g, 2 μeq/g, 1 μeq/g, 0.9 μeq/g, 0.8 μeq/g, 0.7 μeq/g, 0.6 μeq/g, 0.5 μeq/g, 0.4 μeq/g, 0.3 μeq/g, 0.2 μeq/g, 0.1 μeq/g, 0.05 μeq/g, or less than 0.01 μeq/g) of titratable base. In some embodiments, the acetonitrile composition comprises less than 1 μeq/g of titratable base. In some embodiments, the acetonitrile composition comprises less than 0.8 μeq/g of titratable base. In some embodiments, the acetonitrile composition comprises less than 0.6 μeq/g of titratable base. In some embodiments, the acetonitrile composition comprises less than 0.3 μeq/g of titratable base. In some embodiments, the acetonitrile composition comprises from about 0.01 μeq/g to about 10 μeq/g, about 0.01 μeq/g to about 1 μeq/g, about 0.01 μeq/g to about 0.6 μeq/g, or from about 0.01 μeq/g to about 0.03 μeq/g of titratable base.

In some embodiments, the acetonitrile composition comprises a residue after evaporation of less than 0.01% (e.g., less than less than 0.009%, 0.008%, 0.007%, 0.006%, 0.005%, 0.004%, 0.003%, 0.002%, or 0.001%). In some embodiments, the acetonitrile composition comprises a residue after evaporation of less than 0.001% (e.g., less than 0.0009%, 0.0008%, 0.0007%, 0.0006%, 0.0005%, 0.0004%, 0.0003%, 0.0002%, or 0.0001%). In some embodiments, the acetonitrile composition comprises a residue after evaporation of less than 0.0005%. In some embodiments, the acetonitrile composition comprises a residue after evaporation of from 0.0001% to about 0.01%, from about 0.0005% to about 0.005%, from about 0.0005% to about 0.002%, or from about 0.0008% to about 0.002%.

In some embodiments, the purity of the acetonitrile composition may be measured by absorbance. The absorbance may be measured using UV-vis spectroscopy.

In some embodiments, the acetonitrile composition comprises an absorbance at a wavelength of 195 nm of less than 1 AU (e.g., less than 0.9 AU, 0.8 AU, 0.7 AU, 0.6 AU, 0.5 AU, 0.4 AU, 0.3 AU, 0.2 AU, or less than 0.1 AU). In some embodiments, the acetonitrile composition comprises an absorbance at a wavelength of 195 nm of less than 0.9 AU. In some embodiments, the acetonitrile composition comprises an absorbance at a wavelength of 195 nm of less than 0.8 AU. In some embodiments, the acetonitrile composition comprises an absorbance at a wavelength of 195 nm of less than 0.6 AU. In some embodiments, the acetonitrile composition comprises an absorbance at a wavelength of 195 nm of less than 0.5 AU. In some embodiments, the acetonitrile composition comprises an absorbance at a wavelength of 195 nm of from about 0.1 AU to about 1 AU, from about 0.2 AU to about 0.8 AU, from about 0.3 AU to about 0.9 AU, or from about 0.4 AU to about 1 AU.

In some embodiments, the acetonitrile composition comprises an absorbance at a wavelength of 195 nm of less than 0.3 AU (e.g., less than 0.28 AU, 0.25 AU, 0.24 AU, 0.22 AU, 0.2 AU, 0.18 AU, 0.16 AU, 0.15 AU, 0.14 AU, 0.12 AU, 0.1 AU, 0.08 AU, 0.06 AU, or less than 0.05 AU). In some embodiments, the acetonitrile composition comprises an absorbance at a wavelength of 195 nm of less than 0.2 AU. In some embodiments, the acetonitrile composition comprises an absorbance at a wavelength of 195 nm of less than 0.15 AU. In some embodiments, the acetonitrile composition comprises an absorbance at a wavelength of 195 nm of less than 0.1 AU. In some embodiments, the acetonitrile composition comprises an absorbance at a wavelength of 195 nm of from 0.05 AU to about 0.3 AU, from about 0.05 AU to about 0.025 AU, from about 0.05 AU to about 0.02 AU, from about 0.1 AU to about 0.3 AU, or from about 0.1 AU to about 0.2 AU.

In some embodiments, the acetonitrile composition comprises an absorbance at a wavelength of 200 nm of less than 0.2 AU (e.g., less than 0.19 AU, 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, or less than 0.01 AU). In some embodiments, the acetonitrile composition comprises an absorbance at a wavelength of 200 nm of less than 0.15 AU. In some embodiments, the acetonitrile composition comprises an absorbance at a wavelength of 200 nm of less than 0.1 AU. In some embodiments, the acetonitrile composition comprises an absorbance at a wavelength of 200 nm of less than 0.09 AU. In some embodiments, the acetonitrile composition comprises an absorbance at a wavelength of 200 nm of less than 0.08 AU. In some embodiments, the acetonitrile composition comprises an absorbance at a wavelength of 200 nm of less than 0.07 AU. In some embodiments, the acetonitrile composition comprises an absorbance at a wavelength of 200 nm of less than 0.06 AU. In some embodiments, the acetonitrile composition comprises an absorbance at a wavelength of 200 nm of less than 0.05 AU. In some embodiments, the acetonitrile composition comprises an absorbance at a wavelength of 200 nm of less than 0.04 AU. In some embodiments, the acetonitrile composition comprises an absorbance at a wavelength of 200 nm of less than 0.03 AU. In some embodiments, the acetonitrile composition comprises an absorbance at a wavelength of 200 nm of from 0.01 AU to about 0.1 AU, from about 0.01 AU to about 0.09 AU, from about 0.01 AU to about 0.08 AU, from about 0.01 AU to about 0.07 AU, or from about 0.01 AU to about 0.05 AU.

In some embodiments, the acetonitrile composition comprises an absorbance at a wavelength of 210 nm of less than 0.2 AU (e.g., less than 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, 0.01, or less than 0.005 AU). In some embodiments, the acetonitrile composition comprises an absorbance at a wavelength of 210 nm of less than 0.15 AU. In some embodiments, the acetonitrile composition comprises an absorbance at a wavelength of 210 nm of less than 0.1 AU. In some embodiments, the acetonitrile composition comprises an absorbance at a wavelength of 210 nm of less than 0.08 AU. In some embodiments, the acetonitrile composition comprises an absorbance at a wavelength of 210 nm of less than 0.06 AU. In some embodiments, the acetonitrile composition comprises an absorbance at a wavelength of 210 nm of less than 0.05 AU. In some embodiments, the acetonitrile composition comprises an absorbance at a wavelength of 210 nm of less than 0.04 AU. In some embodiments, the acetonitrile composition comprises an absorbance at a wavelength of 210 nm of less than 0.03 AU. In some embodiments, the acetonitrile composition comprises an absorbance at a wavelength of 210 nm of less than 0.02 AU. In some embodiments, the acetonitrile composition comprises an absorbance at a wavelength of 210 nm of less than 0.01 AU. In some embodiments, the acetonitrile composition comprises an absorbance at a wavelength of 210 nm of from 0.005 AU to about 0.1 AU, from about 0.005 AU to about 0.08 AU, from about 0.005 AU to about 0.04 AU, from about 0.005 AU to about 0.03 AU, or from about 0.01 AU to about 0.03 AU.

In some embodiments, the acetonitrile composition comprises an absorbance at a wavelength of 220 nm of less than 0.2 AU (e.g., less than 0.18 AU, 0.17 AU, 0.16 AU, 0.15 AU, 0.14 AU, 0.13 AU, 0.12 AU, 0.11 AU, 0.10 AU, 0.09 AU, 0.08 AU, 0.07 AU, 0.06 AU, 0.05 AU, 0.04 AU, 0.03 AU, 0.02 AU, 0.01, or less than 0.005 AU). In some embodiments, the acetonitrile composition comprises an absorbance at a wavelength of 220 nm of less than 0.15 AU. In some embodiments, the acetonitrile composition comprises an absorbance at a wavelength of 220 nm of less than 0.1 AU. In some embodiments, the acetonitrile composition comprises an absorbance at a wavelength of 220 nm of less than 0.08 AU. In some embodiments, the acetonitrile composition comprises an absorbance at a wavelength of 220 nm of less than 0.06 AU. In some embodiments, the acetonitrile composition comprises an absorbance at a wavelength of 220 nm of less than 0.05 AU. In some embodiments, the acetonitrile composition comprises an absorbance at a wavelength of 220 nm of less than 0.04 AU. In some embodiments, the acetonitrile composition comprises an absorbance at a wavelength of 220 nm of less than 0.03 AU. In some embodiments, the acetonitrile composition comprises an absorbance at a wavelength of 220 nm of less than 0.02 AU. In some embodiments, the acetonitrile composition comprises an absorbance at a wavelength of 220 nm of less than 0.01 AU. In some embodiments, the acetonitrile composition comprises an absorbance at a wavelength of 220 nm of from 0.005 AU to about 0.1 AU, from about 0.005 AU to about 0.08 AU, from about 0.005 AU to about 0.04 AU, from about 0.005 AU to about 0.03 AU, or from about 0.01 AU to about 0.03 AU.

In some embodiments, the acetonitrile composition comprises an absorbance at a wavelength of 230 nm of less than 0.15 AU (e.g., less than 0.14 AU, 0.12 AU, 0.10 AU, 0.09 AU, 0.08 AU, 0.07 AU, 0.06 AU, 0.05 AU, 0.04 AU, 0.03 AU, 0.02 AU, 0.01, 0.005 AU, or less than 0.001 AU). In some embodiments, the acetonitrile composition comprises an absorbance at a wavelength of 230 nm of less than 0.1 AU. In some embodiments, the acetonitrile composition comprises an absorbance at a wavelength of 230 nm of less than 0.05 AU. In some embodiments, the acetonitrile composition comprises an absorbance at a wavelength of 230 nm of less than 0.02 AU. In some embodiments, the acetonitrile composition comprises an absorbance at a wavelength of 230 nm of less than 0.01 AU. In some embodiments, the acetonitrile composition comprises an absorbance at a wavelength of 230 nm of less than 0.005 AU. In some embodiments, the acetonitrile composition comprises an absorbance at a wavelength of 230 nm of less than 0.003 AU. In some embodiments, the acetonitrile composition comprises an absorbance at a wavelength of 230 nm of less than 0.001 AU. In some embodiments, the acetonitrile composition comprises an absorbance at a wavelength of 230 nm of from 0.001 AU to about 0.1 AU, from about 0.001 AU to about 0.08 AU, from about 0.001 AU to about 0.02 AU, from about 0.001 AU to about 0.01 AU, or from about 0.005 AU to about 0.01 AU.

In some embodiments, the acetonitrile composition comprises an absorbance at a wavelength of 240 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 240 nm of less than 0.015 AU. In some embodiments, the acetonitrile composition comprises an absorbance at a wavelength of 240 nm of less than 0.01 AU. In some embodiments, the acetonitrile composition comprises an absorbance at a wavelength of 240 nm of less than 0.009 AU. In some embodiments, the acetonitrile composition comprises an absorbance at a wavelength of 240 nm of less than 0.008 AU. In some embodiments, the acetonitrile composition comprises an absorbance at a wavelength of 240 nm of less than 0.007 AU. In some embodiments, the acetonitrile composition comprises an absorbance at a wavelength of 240 nm of less than 0.006 AU. In some embodiments, the acetonitrile composition comprises an absorbance at a wavelength of 240 nm of less than 0.005 AU. In some embodiments, the acetonitrile composition comprises an absorbance at a wavelength of 240 nm of less than 0.004 AU. In some embodiments, the acetonitrile composition comprises an absorbance at a wavelength of 240 nm of less than 0.003 AU. In some embodiments, the acetonitrile composition comprises an absorbance at a wavelength of 240 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 260 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 260 nm of less than 0.015 AU. In some embodiments, the acetonitrile composition comprises an absorbance at a wavelength of 260 nm of less than 0.01 AU. In some embodiments, the acetonitrile composition comprises an absorbance at a wavelength of 260 nm of less than 0.009 AU. In some embodiments, the acetonitrile composition comprises an absorbance at a wavelength of 260 nm of less than 0.008 AU. In some embodiments, the acetonitrile composition comprises an absorbance at a wavelength of 260 nm of less than 0.007 AU. In some embodiments, the acetonitrile composition comprises an absorbance at a wavelength of 260 nm of less than 0.006 AU. In some embodiments, the acetonitrile composition comprises an absorbance at a wavelength of 260 nm of less than 0.005 AU. In some embodiments, the acetonitrile composition comprises an absorbance at a wavelength of 260 nm of less than 0.004 AU. In some embodiments, the acetonitrile composition comprises an absorbance at a wavelength of 260 nm of less than 0.003 AU. In some embodiments, the acetonitrile composition comprises an absorbance at a wavelength of 260 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 280 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 280 nm of less than 0.015 AU. In some embodiments, the acetonitrile composition comprises an absorbance at a wavelength of 280 nm of less than 0.01 AU. In some embodiments, the acetonitrile composition comprises an absorbance at a wavelength of 280 nm of less than 0.009 AU. In some embodiments, the acetonitrile composition comprises an absorbance at a wavelength of 280 nm of less than 0.008 AU. In some embodiments, the acetonitrile composition comprises an absorbance at a wavelength of 280 nm of less than 0.007 AU. In some embodiments, the acetonitrile composition comprises an absorbance at a wavelength of 280 nm of less than 0.006 AU. In some embodiments, the acetonitrile composition comprises an absorbance at a wavelength of 280 nm of less than 0.005 AU. In some embodiments, the acetonitrile composition comprises an absorbance at a wavelength of 280 nm of less than 0.004 AU. In some embodiments, the acetonitrile composition comprises an absorbance at a wavelength of 280 nm of less than 0.003 AU. In some embodiments, the acetonitrile composition comprises an absorbance at a wavelength of 280 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition comprises an absorbance at a wavelength of 400 nm of less than 0.02 AU (e.g., less than 0.019 AU, 0.018 AU, 0.017 AU, 0.016 AU, 0.015 AU, 0.014 AU, 0.013 AU, 0.012 AU, 0.011 AU, 0.01 AU, 0.009 AU, 0.008 AU, 0.007 AU, 0.006 AU, 0.005 AU, 0.004 AU, 0.003 AU, 0.002 AU, or less than 0.001 AU). In some embodiments, the acetonitrile composition comprises an absorbance at a wavelength of 400 nm of less than 0.015 AU. In some embodiments, the acetonitrile composition comprises an absorbance at a wavelength of 400 nm of less than 0.01 AU. In some embodiments, the acetonitrile composition comprises an absorbance at a wavelength of 400 nm of less than 0.009 AU. In some embodiments, the acetonitrile composition comprises an absorbance at a wavelength of 400 nm of less than 0.008 AU. In some embodiments, the acetonitrile composition comprises an absorbance at a wavelength of 400 nm of less than 0.007 AU. In some embodiments, the acetonitrile composition comprises an absorbance at a wavelength of 400 nm of less than 0.006 AU. In some embodiments, the acetonitrile composition comprises an absorbance at a wavelength of 400 nm of less than 0.005 AU. In some embodiments, the acetonitrile composition comprises an absorbance at a wavelength of 400 nm of less than 0.004 AU. In some embodiments, the acetonitrile composition comprises an absorbance at a wavelength of 400 nm of less than 0.003 AU. In some embodiments, the acetonitrile composition comprises an absorbance at a wavelength of 400 nm of from 0.001 AU to about 0.01 AU, from about 0.001 AU to about 0.009 AU, from about 0.001 AU to about 0.008 AU, from about 0.001 AU to about 0.007 AU, or from about 0.001 AU to about 0.005 AU.

In some embodiments, the acetonitrile composition has a color (APHA) of less than 20 (e.g., less than 19, 18, 17, 16, 15, 14, 13, 12, 11, 10, 9, 8, 7, 6, 5, 4, 3, 2, or less than 1). In some embodiments, the acetonitrile composition has a color (APHA) of less than 15. In some embodiments, the acetonitrile composition has a color (APHA) of less than 10. In some embodiments, the acetonitrile composition has a color (APHA) of less than 5. In some embodiments, the acetonitrile composition has a color (APHA) of from about 1 to about 20, of from about 1 to about 15, of from about 1 to about 10, of from about 5 to about 15, or from about 5 to about 10.

The color of the acetonitrile composition may be measured according to the American Public Health Association (APHA) color scale.

In some embodiments, the acetonitrile composition provided herein is an oligonucleotide synthesis grade acetonitrile composition.

In some embodiments, oligonucleotide synthesis grade acetonitrile composition comprises purity of at least 99.8%. In some embodiments, oligonucleotide synthesis grade acetonitrile composition comprises a density of from about 0.780 g/mL to about 0.783 g/mL at 20° C. In some embodiments, oligonucleotide synthesis grade acetonitrile composition comprises a refractive index of from about 1.343 to about 1.346 at 20° C. In some embodiments, oligonucleotide synthesis grade acetonitrile composition comprises less than 10 ppm of water. In some embodiments, oligonucleotide synthesis grade acetonitrile composition comprises an acidity (as CH₃COOH) of less than 0.001%. In some embodiments, oligonucleotide synthesis grade acetonitrile composition comprises a residue after evaporation of less than 0.001%. In some embodiments, oligonucleotide synthesis grade acetonitrile composition comprises an absorbance at a wavelength of 200 nm of less than 0.05 AU. In some embodiments, oligonucleotide synthesis grade acetonitrile composition comprises an absorbance at a wavelength of 210 nm of less than 0.03 nm. In some embodiments, oligonucleotide synthesis grade acetonitrile composition comprises an absorbance at a wavelength of 220 nm of less than 0.02 nm. In some embodiments, oligonucleotide synthesis grade acetonitrile composition comprises an absorbance at a wavelength of 230 nm of less than 0.01 nm. In some embodiments, oligonucleotide synthesis grade acetonitrile composition comprises an absorbance at a wavelength of 240 nm of less than 0.005 AU. In some embodiments, oligonucleotide synthesis grade acetonitrile composition comprises an absorbance at a wavelength of 260 nm of less than 0.005 AU. In some embodiments, oligonucleotide synthesis grade acetonitrile composition comprises an absorbance at a wavelength of 280 nm of less than 0.005 AU. In some embodiments, oligonucleotide synthesis grade acetonitrile composition comprises an absorbance at a wavelength of 400 nm of less than 0.005 AU. In some embodiments, oligonucleotide synthesis grade acetonitrile composition comprises a color (APHA) of less than 10.

In some embodiments, the acetonitrile composition provided herein is an ACS grade acetonitrile composition.

In some embodiments, ACS grade acetonitrile composition comprises a purity of at least 99.5%. In some embodiments, ACS grade acetonitrile composition comprises less than 3000 ppm (0.3%) water. In some embodiments, ACS grade acetonitrile composition comprises a residue after evaporation of less than 0.005%. In some embodiments, ACS grade acetonitrile composition comprises a color (APHA) of less than 10. In some embodiments, ACS grade acetonitrile composition comprises less than 8 μeq/g of titratable acid. In some embodiments, ACS grade acetonitrile composition comprises less than 0.6 μeq/g of titratable base. In some embodiments, the identity of ACS grade acetonitrile composition is confirmed by infrared spectroscopy.

In some embodiments, the acetonitrile composition provided herein is an HPLC grade acetonitrile composition.

In some embodiments, the HPLC grade acetonitrile composition comprises a purity of at least 99.9%. In some embodiments, the HPLC grade acetonitrile composition comprises an absorbance at a wavelength of 254 nm of less than 0.01 AU. In some embodiments, the HPLC grade acetonitrile composition comprises an absorbance at a wavelength of 220 nm of less than 0.02 AU. In some embodiments, the HPLC grade acetonitrile composition comprises an absorbance at a wavelength of 210 nm of less than 0.04 AU. In some embodiments, the HPLC grade acetonitrile composition comprises an absorbance at a wavelength of 205 nm of less than 0.05 AU. In some embodiments, the HPLC grade acetonitrile composition comprises an absorbance at a wavelength of 200 nm of less than 0.07 AU. In some embodiments, the HPLC grade acetonitrile composition comprises an absorbance at a wavelength of 295 nm of less than 0.15 AU. In some embodiments, the HPLC grade acetonitrile composition comprises an absorbance at a wavelength of 190 nm of less than 1 AU. In some embodiments, the HPLC grade acetonitrile composition comprises less than 8 μeq/g of titratable acid. In some embodiments, the HPLC grade acetonitrile composition comprises less than 0.6 μeq/g of titratable base. In some embodiments, the HPLC grade acetonitrile composition comprises less than 100 ppm (0.01%) water. In some embodiments, the HPLC grade acetonitrile composition comprises a residue after evaporation of less than 0.0001% (less than 1 ppm).

In some embodiments, the acetonitrile composition provided herein is an LC/MS grade acetonitrile. In some embodiments, the LC/MS grade acetonitrile composition has a purity of at least 99.8%.

In some embodiments, the acetonitrile composition is oligonucleotide synthesis grade, ACS grade, HPLC grade, LC/MS grade, and/or pharmaceutical grade acetonitrile. In some embodiments, such a grade acetonitrile composition comprises a purity of at least 99.8%. In some embodiments, pharmaceutical grade acetonitrile composition comprises less than 2000 ppm water (0.2%).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Examples

### Example 1: Method of Preparing Bio-Acetonitrile

Acetonitrile may be prepared according to the scheme in FIG. 1. Ethanol was provided (106) and fermented to acetic acid according to vinegar or white vinegar production methods (102). The method includes semi-fed batch aerobic fermentation operated in a cycle including two modes, start-up, or cycle. In start-up, a tank was filled with water, starter culture, and fed ethanol continuously until the acetic acid concentration reached 20 wt %. At this point, 60% of the fermenter was dropped to the well and the system was in cycle mode. The remaining 40% was retained and the fermenter was filled with water and the ethanol feeding process began again. The fermenters were continuously aerated at all times. Acetic acid was pumped from the fermenter to the acetic acid well. The acetic acid was pumped to the filter tank where it was recycled through the filter system until it was concentrated. Filtration removes rejected biomass. An example scheme for production of acetic acid from fermentation of ethanol is shown in FIG. 5.

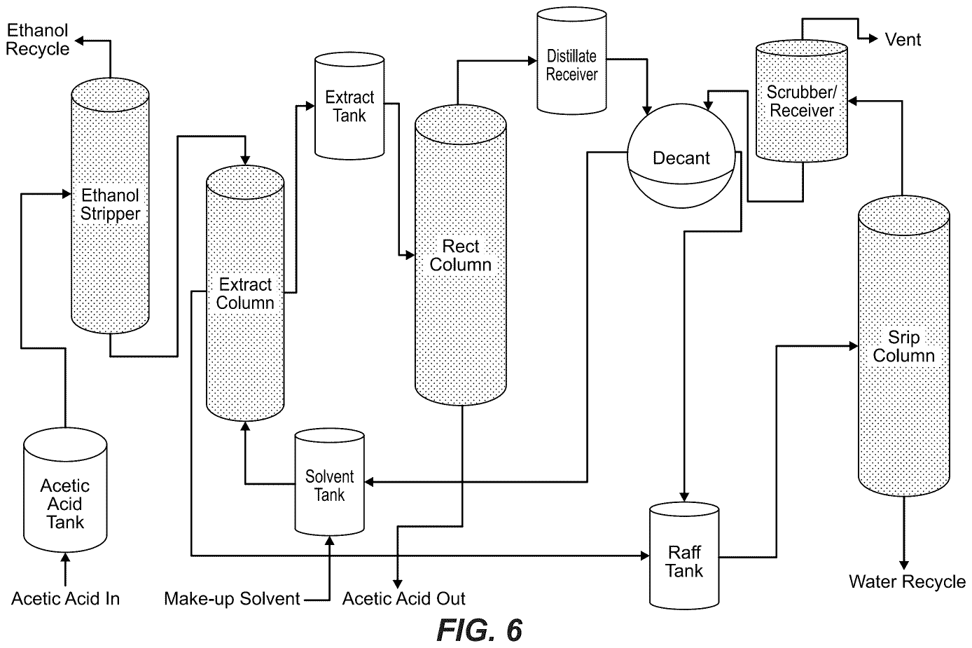

The filtered acetic acid was fed to an acetic acid purification section (103), FIG. 6. The acetic acid purification consists of a hybrid-extraction-distillation train. The first step was stripping of the acetic acid to remove ethanol (ethanol stripper of FIG. 6). This ethanol can be optionally recycled to the acetic acid fermentation process (107). The stripped acetic acid was then fed to an extraction column where it interacts with a methyl tert-butyl ether (MTBE) solvent stream (108) (extract column of FIG. 6). The MTBE-rich extract was fed to the rectification column from which a pure acetic acid stream was generated (rect. column of FIG. 6). The water-rich raffinate was fed to the water stripping column, where residual MTBE was vaporized and removed from the water. The water, which leaves the water stripping column (strip column of FIG. 6), may be recycled back to the fermentation section (107). The distillate of both the rectification column and the water stripping column were combined, cooled, and sent to a decanter where the organic and aqueous phases were separated and sent to the extraction column and the water stripping column, respectively. The water recovered from the water stripping column optionally either underwent wastewater treatment (110) or was recycled for use during the acetic acid fermentation.

The resulting (e.g., glacial) acetic acid was preheated, mixed, and fed to the reactor (104) with ammonia (109) and optionally the recycled ammonia stream (112). The reactor was a fixed bed catalyst operated at a temperature between 280° C.-400° C. (e.g., 290° C.-320° C.) and a pressure of 1-10 bar (e.g., 1-4 bar). The reaction was endothermic, and the reactor must be heated to maintain favorable reaction temperatures. In some instances, side reactions become more pronounced at higher temperatures, and reactor fouling occurs at lower temperatures due to incomplete reaction. The reaction of the acetic acid and ammonia is shown in FIG. 3. The resulting crude reactor product contains ammonia, water, acetonitrile, acetone, carbon dioxide, and minor impurities.

The crude reactor product was purified (105). Ammonia was distilled off from the crude reactor product via a water scrubbing distillation column and the nominally pure ammonia stream was optionally recycled to the reactor feed (112). The crude reactor product was mixed with an oxidant, such as potassium permanganate, under acidic, neutral, or basic conditions. Oxidation of acetone and other impurities may allow for further purification of the acetonitrile. Excess oxidant was quenched (e.g., with sodium thiosulfate), the solution was returned to neutral pH (e.g., by adding sulfuric acid if the oxidation reaction was conducted under basic conditions), and solids formed during the reaction were filtered. The oxidized, quenched, neutralized, and filtered crude product was fed to a modified pressure swing distillation for water (and impurities) and acetonitrile separation.

During the acetonitrile purification (105), the crude reactor product was cooled and fed to an ammonia recovery column, the ammonia and carbon dioxide are the distillate products. The ammonia and carbon dioxide vapor were scrubbed in a multi-stage water scrubbing column before the nominally pure ammonia was recycled to the reactor feed. The aqueous carbon dioxide stream was partially bled off as waste and recycled to the scrubber. The water, acetonitrile, and acetone was fed to a continuously stirred tank reactor, where an oxidizing agent or catalysts such as potassium permanganate was fed to the system. This reactor has an adequately long residence time (as little as 20 minutes or up to 2 hours or more) for acetone and other impurities (e.g., unsaturated nitriles) to fully react. The reactor effluent was fed to a distillation column which separates the oxidation reaction products from the crude reactor product. The purified reactor product was fed to the pressure swing distillation apparatus. First, this stream enters the low-pressure column, which operates at atmospheric pressure or below atmospheric pressure. There is a secondary feed stream to this column which is the azeotrope recycled from the high-pressure column. The bottoms product of the low-pressure column is nominally pure water. The distillate of the low-pressure column is near the water and acetonitrile azeotrope, although this can be distorted due to the presence of impurities (primarily acetone and ammonia). The low-pressure column distillate was chilled, compressed to higher pressure, and fed to the high-pressure column. The bottoms of the high-pressure column are nominally pure acetonitrile. The distillate of the high-pressure column is the higher-pressure water and acetonitrile azeotrope, with additional impurities (primarily acetone and ammonia). The high-pressure distillate was fed to a water stripping column which purges acetone and any residual ammonia and/or carbon dioxide (or other volatiles) in the distillate (111). The bottoms of the water stripping column was nominally water and acetonitrile at the high-pressure azeotrope, which was recycled to the low-pressure column.

The nominally pure acetonitrile was then polished to meet requisite purity specifications (105). Purity specifications may include technical grade, laboratory grade, USP grade, ACS grade, pharma grade, or oligonucleotide synthesis grade. This was accomplished via two sequential polishing steps. The acetonitrile was passed through an activated carbon bed which removes the aromatics (below ˜1 ppm). This stream was passed through a bed of 3A molecular sieves to remove water. Finally, any remaining water and trace polar impurities were removed via an activated alumina column to below 10 ppm. The polished acetonitrile (101) was (e.g., immediately) packaged and stored.

### Example 2: Method of Preparing Bio-Acetonitrile

Acetonitrile may be prepared according to the scheme in FIG. 2. Acetic acid may be provided (202) as produced by any suitable method. The acetic acid may be purified or require purification in subsequent steps.

The acetic acid was fed to an acetic acid purification section (203), FIG. 6. The acetic acid purification consists of a hybrid-extraction-distillation train. The first step was stripping of the acetic acid to remove ethanol (ethanol stripper of FIG. 6). The stripped acetic acid was then fed to an extraction column where it interacted with a methyl tert-butyl ether (MTBE) solvent stream (208) (extract column of FIG. 6). The MTBE-rich extract was fed to the rectification column from which a pure acetic acid stream is generated (rect. column of FIG. 6). The water-rich raffinate was fed to the water stripping column, where residual MTBE was vaporized and removed from the water. The distillate of both the rectification column and the water stripping column were combined, cooled, and sent to a decanter where the organic and aqueous phases were separated and sent to the extraction column and the water stripping column, respectively. The water recovered from the water stripping column optionally either underwent wastewater treatment or was recycled for use during the acetic acid fermentation.

The resulting (e.g., glacial) acetic acid was preheated, mixed, and fed to the reactor (204) with ammonia (208) and optionally the recycled ammonia stream (211). The reactor was a fixed bed catalyst operated at a temperature between 280° C.-400° C. (e.g., 290° C.-320° C.) and a pressure of 1-10 bar (e.g., 1-4 bar). The reaction was endothermic, and the reactor must be heated to maintain favorable reaction temperatures. In some instances, side reactions become more pronounced at higher temperatures, and reactor fouling occurs at lower temperatures due to incomplete reaction. The reaction of the acetic acid and ammonia is shown in FIG. 3. The resulting crude reactor product contains ammonia, water, acetonitrile, acetone, carbon dioxide, and minor impurities.

The crude reactor product was purified (205). Ammonia was distilled off from the crude reactor product via a water scrubbing distillation column and the nominally pure ammonia stream was optionally recycled to the reactor feed (211). The crude reactor product was mixed with an oxidant, such as potassium permanganate, under acidic, neutral, or basic conditions. Oxidation of acetone and other impurities may allow for further purification of the acetonitrile. Excess oxidant was quenched (e.g., with sodium thiosulfate), the solution was returned to neutral pH (e.g., by adding sulfuric acid if the oxidation reaction was conducted under basic conditions), and solids formed during the reaction were filtered. The oxidized, quenched, neutralized, and filtered crude product was fed to a modified pressure swing distillation for water (and impurities) and acetonitrile separation.

During the acetonitrile purification (205), the crude reactor product was cooled and fed to an ammonia recovery column, the ammonia and carbon dioxide are the distillate products. The ammonia and carbon dioxide vapor was scrubbed in a multi-stage water scrubbing column before the nominally pure ammonia was recycled to the reactor feed. The aqueous carbon dioxide stream was partially bled off as waste and recycled to the scrubber. The water, acetonitrile, and acetone were fed to a continuously stirred tank reactor, where an oxidizing agent or catalysts such as potassium permanganate was fed to the system. This reactor had an adequately long residence time (as little as 20 minutes or up to 2 hours or more) for acetone and other impurities (e.g., unsaturated nitriles) to fully react. The reactor effluent was fed to a distillation column which separates the oxidation products from the crude reactor product. The distillate of this column was fed to the pressure swing distillation apparatus. First, this stream enter ed the low-pressure column, which operates at or below 1 atm(g). There is a secondary feed stream to this column which is the azeotrope recycled from the high-pressure column. The bottoms product of the low-pressure column is nominally pure water. The distillate of the low-pressure column was near the water and acetonitrile azeotrope, although the exact composition can be distorted due to the presence of impurities (primarily acetone). The low-pressure column distillate was chilled, compressed to higher pressure, and fed to the high-pressure column. The bottoms of the high-pressure column are nominally pure acetonitrile. The distillate of the high-pressure column was the higher-pressure water and acetonitrile azeotrope, with additional impurities (primarily acetone). The high-pressure distillate was fed to a water stripping column which purges acetone and any residual ammonia and/or carbon dioxide (or other volatiles) in the distillate (210). The bottoms of the water stripping column was nominally water and acetonitrile at the high-pressure azeotrope, which was recycled to the low-pressure column.

The nominally pure acetonitrile was then polished to meet requisite purity specifications (105). Purity specifications may include technical grade, laboratory grade, USP grade, ACS grade, pharma grade, or oligonucleotide synthesis grade. This was accomplished via two sequential polishing steps. The acetonitrile was passed through an activated carbon bed which removes the aromatics (below ˜1 ppm) and a molecular sieve bed to remove the majority of the water. Finally, any remaining water and other polar impurities were removed via an activated alumina column to below 10 ppm. The polished acetonitrile (201) was (e.g., immediately) packaged and stored.

### Example 3: Pilot Scale Nitrilation

Liquid glacial acetic acid was pumped into the reactor system at ambient temperature, then mixed with nitrogen and preheated to 270° C., and then co-fed with preheated ammonia into a 5 L packed bed reactor held between 300° C.-320° C. at elevated pressure. The reactor was packed with 1.8 kg of anatase TiO₂ (⅛″ pellets). Downstream of the reactor, a side stream of reactor product was analyzed via FTIR to quantify reaction yields and impurity levels. The remaining reactor product was cooled, generating a liquid crude product stream composed primarily of acetonitrile and water, as well as a waste vapor stream consisting primarily of nitrogen and ammonia. Liquid crude products were further analyzed via GC-MS to validate reaction yields and impurity levels. Several example reactor runs with reactor pressure, reactant flow rates, and resultant reactor products are included in Table 1.

TABLE 1
Examples of reactor conditions and feed rates as well as acetonitrile yields and impurity content.

| | | | | | Acetic | | | | | | |
|---|---|---|---|---|---|---|---|---|---|---|---|
| | | NH3: | | | acid | NH3 | | Aceto- | produced | produced | Acetonitrile |
| | | Acetic | Reactor | N2 | flow | flow | Residence | nitrile | acetonitrile: | acetonitrile: | production |
| | WHSV | acid | Pressure | Flowrate | rate | rate | Time | Yield | acetamide | acetone | rate |
| No. | (hr−1) | (mol/mol) | (bar(g)) | (SLPM) | (g/hr) | (SLPM) | (s) | (%) | (mol/mol) | (mol/mol) | (kg/hr) |
| 1 | 0.30 | 9 | 1.8 | 10 | 566 | 32 | 2.8 | 63% | 54 | 6 | 0.24 |
| 2 | 0.20 | 13 | 1.5 | 20 | 351 | 28 | 2.1 | 70% | 25 | 8 | 0.17 |
| 3 | 0.30 | 6 | 1.5 | 20 | 566 | 21 | 2.4 | 61% | 69 | 4 | 0.24 |
| 4 | 0.30 | 12 | 1.2 | 15 | 566 | 42 | 1.4 | 73% | 21 | 10 | 0.28 |
| 5 | 0.45 | 12 | 1.5 | 10 | 850 | 63 | 1.3 | 86% | 27 | 34 | 0.50 |
| 6 | 0.45 | 9 | 1.5 | 20 | 850 | 48 | 1.5 | 74% | 36 | 13 | 0.43 |
| 7 | 0.45 | 9 | 1.2 | 20 | 850 | 48 | 1.2 | 82% | 45 | 19 | 0.48 |
| 8 | 0.45 | 9 | 1.2 | 10 | 850 | 48 | 1.3 | 79% | 30 | 14 | 0.46 |
| 9 | 0.45 | 9 | 0.9 | 10 | 850 | 48 | 1.0 | 95% | 42 | 25 | 0.55 |
| 10 | 0.64 | 9 | 0.9 | 10 | 1155 | 65 | 0.8 | 90% | 47 | 33 | 0.71 |

Many sample runs were completed, summarized above in Table 1. In Run 5, glacial acetic acid was fed at a rate of 0.85 kg/hr, nitrogen at 10 SLPM, and ammonia at 63 SLPM. The reactor was held at 1.5 bar(g). At these conditions, reactor residence time was 1.3 seconds and WHSV=0.45 hr⁻¹. Reaction yield of acetic acid to acetonitrile was observed to be 86% at these conditions, with an acetonitrile:acetone molar ratio of 27 mol/mol and acetonitrile:acetamide molar ratio of 34 mol/mol in the product stream. Acetonitrile production was 0.50 kg/hr.

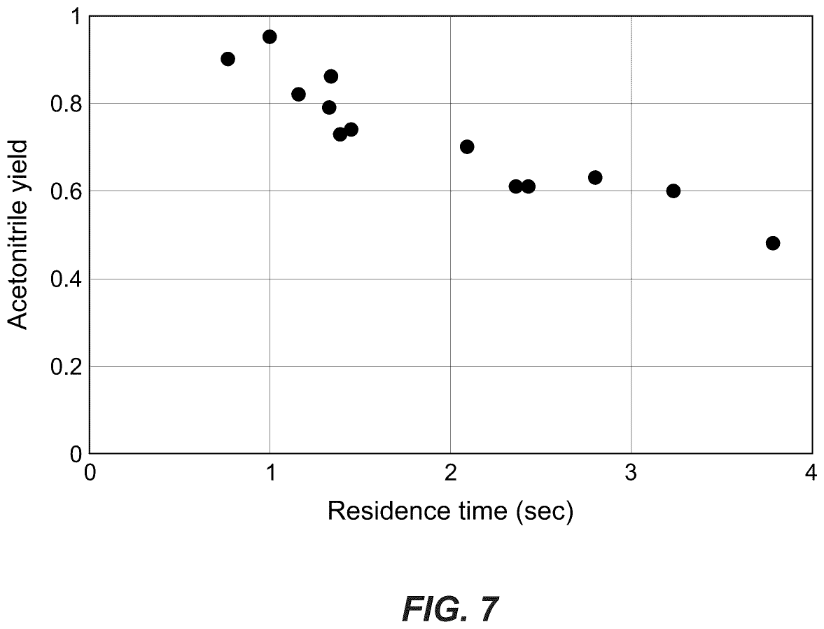

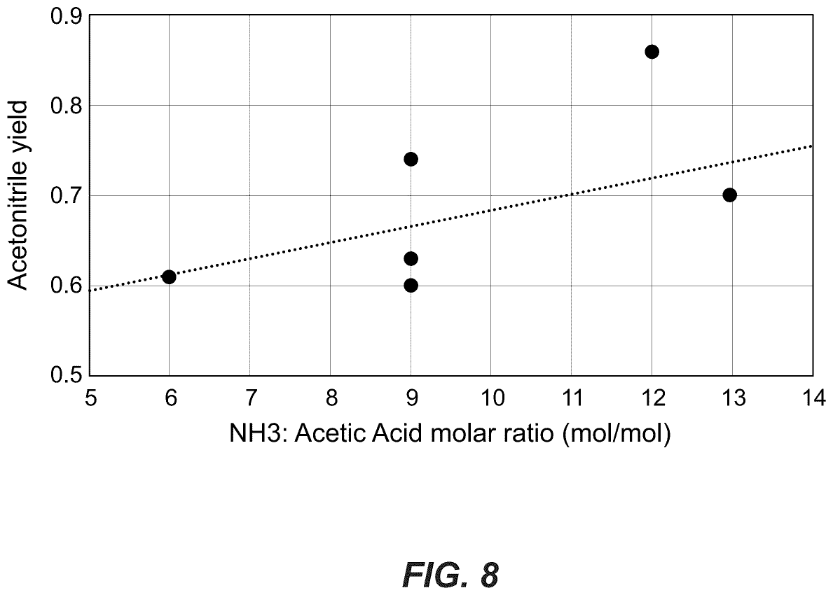

In Run 5, and in general in the results, it was observed that at higher pressures, ammonia:acetic acid ratio has the greatest impact on acetonitrile yield, while at lower pressures residence time plays a greater role. Overall, yield is optimized at low pressure, high ammonia:acetic acid ratio, and low residence time. These effects are shown in FIG. 7 & FIG. 8.

FIG. 7 shows acetonitrile yield as a function of reactor residence time in a 5 L packed bed reactor loaded with 1.8 kg TiO2-anatase. FIG. 8 shows acetonitrile yield as a function of ammonia:acetic acid molar ratio in the feed.

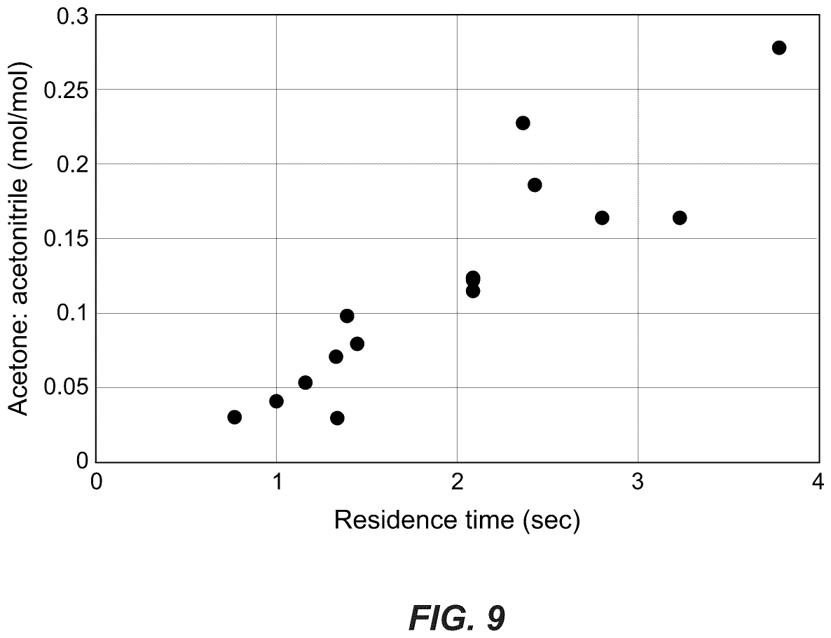

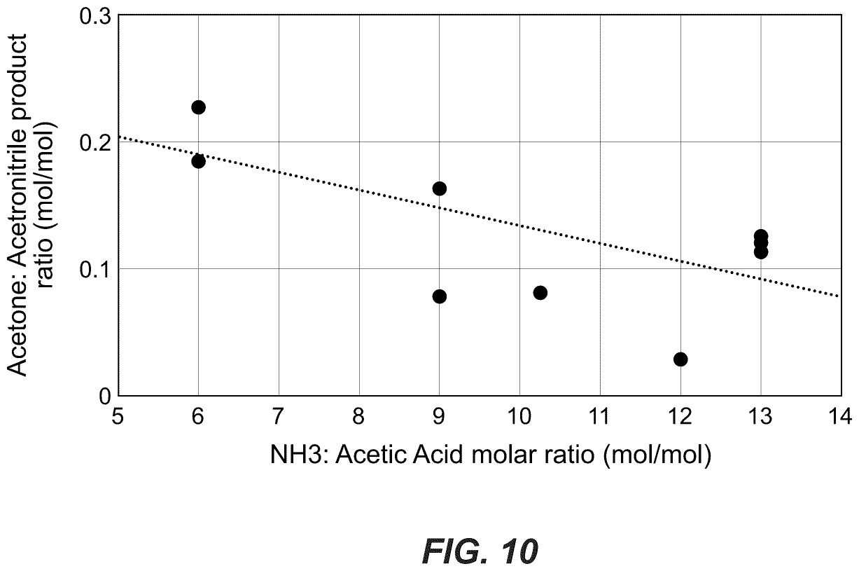

Product purity was also dependent on ammonia:acetic acid ratio and residence time. At low pressures, residence time has the greatest impact on purity with lower residence times resulting in a purer product. At higher pressures, both residence time and ammonia:acetic acid ratio have significant effects, with higher ammonia:acetic acid ratios producing less acetone and acetamide. These effects can be seen on FIG. 9 & FIG. 10.

FIG. 9 shows acetone:acetonitrile molar ratio in the product stream as a function of reactor residence time in a 5 L packed bed reactor loaded with 1.8 kg TiO2-anatase. FIG. 10 shows acetone:acetonitrile molar ratio in the product stream as a function of ammonia:acetic acid molar ratio in the reactor feed.

Provided herein, in some embodiments, are methods of making acetonitrile (and compositions comprising acetonitrile), also referred to herein as “bio-based acetonitrile.” The methods provided herein are environmentally friendly and less toxic alternatives to the standard propylene ammoxidation (SOHIO) process, which may allow for preparation of acetonitrile at high levels of purity (e.g., oligonucleotide synthesis quality or pharmaceutical grade quality) with less energy inputs and lower hazards to operator health and the environment. In some embodiments, the methods herein provide for the use of 100% bio-based feedstocks, allowing for a more ecologically sustainable method for preparation of acetonitrile. Additionally, the methods herein are decoupled from production of other compounds (e.g., acrylonitrile) and provide for direct preparation of acetonitrile without generation of explosive hazards in the process or release of toxic byproducts, such as hydrogen cyanide.

### Example 4: Pilot Scale Reactor Sample Purification Columns

Several additional Examples were run to illustrate the purification results of the acetonitrile production methods discussed herein.

For instance, in Example 4, the crude product, including acetonitrile, was run through a series of distillation steps, then passed through columns to qualify reagent-grade acetonitrile and HPLC-grace acetonitrile.

First, in an initial distillation step, a reactor crude product was initially distilled, and distillates were recovered. The reactor crude product included 158 g/L ammonia, 5 g/L acetone, 5 g/L carbon dioxide, 1 g/L 3-methyl-2-butenenitrile, 77 g/L acetonitrile, and water. The liquid parts of each fraction were recombined with the distillation pot liquid to produce a solution which was then diluted with water, basified with potassium hydroxide, oxidized with potassium permanganate, neutralized with sulfuric acid, then distilled. Next, these distillates were recovered, then diluted with reverse osmosis water, basified with potassium hydroxide, oxidized with potassium permanganate, quenched with sodium thiosulfate, and neutralized with sulfuric acid. The oxidized solution was distilled. The distillates were combined with potassium carbonate to induce phase separation where the phases were saturated with potassium carbonate. The bottom phase was discarded and additional potassium carbonate was added to the top (organic) phase. After settling overnight, the organic phase was distilled.

The distillates were combined and passed through columns of activated carbon, 3A molecular sieves, and activated alumina, neutral. Prior to passing the sample, the adsorbents were calcined overnight at 360° C. and cooled to room temperature under flowing grade zero air, then transferred immediately to the adsorption column. HPLC-grade acetonitrile was passed through the bed materials, then the combined distillates, then HPLC-grade acetonitrile again. Column fractions containing the distillates showed dramatically reduced absorbance in the UV compared to the pre-column distillates. Column fractions containing the distillates were >99.8% pure by GC.

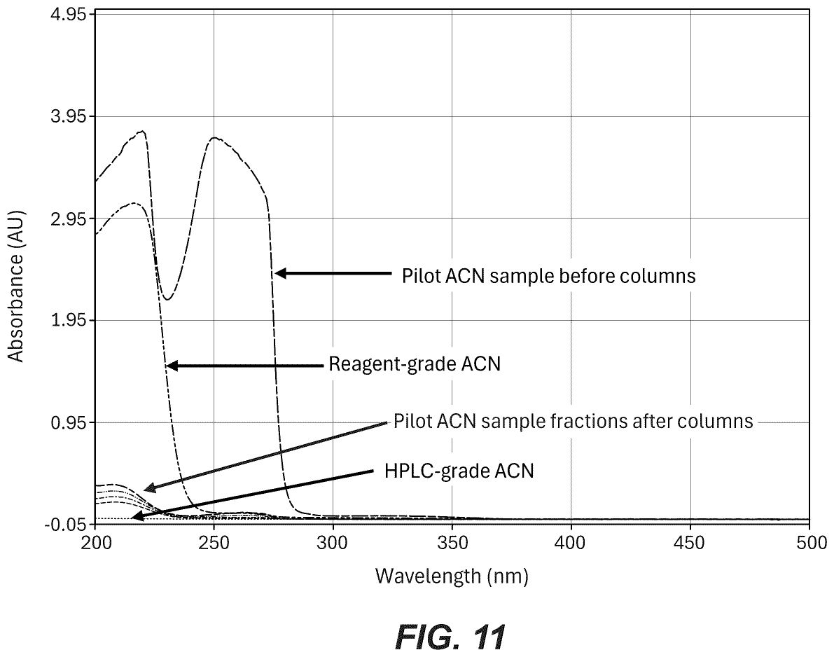

The results of Example 4 are shown in FIG. 11. In FIG. 11, different fractions can be seen, such as pilot acetonitrile samples before columns, reagent grade acetonitrile, pilot acetonitrile sample fractions after columns, and HPLC grade acetonitrile.

### Example 5: Pilot Scale Reactor Sample Purification Fractions

In Example 5, a reactor crude product was distilled, oxidized, and fractions were produced. Specifically, reactor crude product containing 158 g/L ammonia, 5 g/L acetone, 5 g/L carbon dioxide, 1 g/L 3-methyl-2-butenenitrile, 77 g/L acetonitrile, and water was distilled. Distillates were recovered; some were biphasic, and some biphasic fractions precipitated solid ammonium bicarbonate. The liquid parts of each fraction were combined and diluted with reverse osmosis water to produce a solution. This solution was basified with potassium hydroxide and oxidized with potassium permanganate, then neutralized with sulfuric acid and distilled. During distillation, of distillates were recovered across three fractions, with the following compositions:

TABLE 2
Fractions of Example 5.

| | Fraction | | | | Aceto- | 3-methyl-2- |
|---|---|---|---|---|---|---|
| Sample | Mass | NH₃ | H₂O | Acetone | nitrile | butenenitrile |
| Fraction 1 | 16.6 | 0.0 | 157.2 | 38.4 | Balance | 0.0 |
| Fraction 2 | 16.8 | 0.0 | 167.4 | 14.0 | Balance | 0.0 |
| Fraction 3 | 17.7 | 0.0 | 458.1 | 2.1 | Balance | 0.0 |

### Example 6: Pilot Scale Reactor Sample Purification with Acetone Complexation

In Example 6, reactor crude product containing 158 g/L ammonia, 5 g/L acetone, 5 g/L carbon dioxide, 1 g/L 3-methyl-2-butenenitrile, 77 g/L acetonitrile, and water was distilled. Distillates were recovered; some were biphasic and some biphasic fractions precipitated solid ammonium bicarbonate. The liquid parts of each fraction were combined with reverse osmosis water to produce a solution with the following composition: 24 g/L ammonia, 23 g/L acetone, 0.8 g/L 3-methyl-2-butenenitrile, 218 g/L acetonitrile, and water.

A portion of these combined and diluted distillates were neutralized with sulfuric acid, adjusted to pH 6 with potassium hydroxide, and distilled again. During distillation, 42.3 g of distillates were recovered and combined and diluted with reverse osmosis water to form a solution of the following composition: 0 g/L ammonia, 21 g/L acetone, 0.8 g/L 3-methyl-2-butenenitrile, 211 g/L acetonitrile, and water.

The crude and neutralized distillates were separately subjected to acetone complexation with sodium bisulfite. To individual vials were added 10 g distillate sample and either 2.0 or 2.5 g sodium bisulfite (e.g. NaHSO₃). Upon dissolution of NaHSO₃, the mixtures became biphasic. The vials were stirred at 30° C., 40° C., or 50° C. and sampled at 30 min and 60 min. Gas chromatography analysis showed that acetone concentration in the organic phase was decreased from 21-23 g/L to as low as 1.4 g/L under some conditions. This conversion was due to both acetone-bisulfite complexation, which pulled acetone into the aqueous phase, and acetone condensation with acetonitrile, forming 3-methyl-2-butenenitrile that remained in the organic phase. Acetonitrile partitioning into the aqueous phase ranged from 15% to 65% of the feed solution.

Additionally, a mock mixture of sodium bisulfite was tested. For this test, To eight individual vials were added 0.035 g acetone, 1.5-8.2 g acetonitrile, and 0-8.1 g water to achieve three different compositions. To each vial was added 0.26, 0.77, or 1.28 g NaHSO₃ to achieve eight different compositions. The vials were reacted at either 30° C., 40° C., or 50° C. for 60 min. Some of the samples became biphasic. Samples with no water addition did not completely dissolve the NaHSO₃ and showed low acetone conversion. Samples with the highest water addition were not biphasic, and had no second phase to pull complexed acetone away from the acetonitrile. The biphasic samples showed acetone conversion, with the sample at 30° C. demonstrating the highest value at 95.8% acetone conversion. This same sample showed high acetonitrile retention in the organic phase. (e.g., retention being the ratio of concentration at 60 min to initial concentration, thus ratios higher than 100% are possible if the organic phase is richer in acetonitrile than the initial mixture.)

The acetonitrile conversion and retention from these tests are summarized in Table 3.

TABLE 3
Example 6 results.

| | | | | | | | | Aceto- |
|---|---|---|---|---|---|---|---|---|
| | | Aceto- | | | Temper- | | Acetone | nitrile |
| | Acetone | nitrile | Water | NaHSO3 | ature | | Conversion | Retention |
| Run | (g) | (g) | (g) | (g) | (° C.) | Biphasic? | (%) | (%) |
| 1 | 0.031 | 8.227 | 0.000 | 0.265 | 30 | No | 0% | 97% |
| 2 | 0.033 | 8.236 | 0.000 | 0.768 | 40 | No | 0% | 98% |
| 3 | 0.043 | 8.244 | 0.000 | 1.280 | 50 | No | 6% | 96% |
| 5 | 0.037 | 4.905 | 4.066 | 0.770 | 50 | Yes | 64% | 137% |
| 6 | 0.037 | 4.891 | 4.066 | 1.279 | 30 | Yes | 96% | 123% |
| 7 | 0.039 | 1.537 | 8.128 | 0.264 | 50 | No | 0% | 113% |
| 8 | 0.034 | 1.535 | 8.136 | 0.774 | 30 | No | 36% | 77% |
| 9 | 0.038 | 1.537 | 8.135 | 1.278 | 40 | No | 17% | 88% |

### Example 7: Pilot Scale Reactor Sample Purification with Multiple Samples

In Example 7, a reactor crude product containing 104 g/L ammonia, 3 g/L acetone, ˜2 g/L of carbon dioxide, 0.1 g/L 3-methyl-2-butenenitrile, 0.9 g acetamide, 26 g/L acetonitrile, and water was distilled. Distillates were recovered in three fractions. The fractions showed the following compositions in g/L:

TABLE 4
Example 7 Sample 1 Fractions.

| | | | | | 3-methyl-2- |
|---|---|---|---|---|---|
| | NH₃ | H₂O | Acetone | Acetonitrile | butenenitrile |
| Fraction 1 | 119.7 | balance | 16.9 | 205.6 | 3.3 |
| Fraction 2 | 126.9 | balance | 8.7 | 172.6 | 0.9 |
| Fraction 3 | 113.0 | balance | 3.2 | 148.7 | 0.3 |
| Pot | 21.5 | balance | 0.1 | 4.7 | 0.0 |

A second sample of reactor crude product was distilled, and distillates were recovered across eight fractions. The fractions showed the following compositions in g/L:

TABLE 5
Example 7 Sample 2 Fractions.

| | Fraction | | | | Aceto- | 3-methyl-2- |
|---|---|---|---|---|---|---|
| Sample | Mass | NH3 | H2O | Acetone | nitrile | butenenitrile |
| Fraction 1 | 15.6 | 43.7 | 152.9 | 60.9 | balance | 4.5 |
| Fraction 2 | 16.6 | 41.9 | 163.0 | 58.5 | balance | 5.2 |
| Fraction 3 | 15.9 | 26.7 | 111.0 | 65.5 | balance | 4.6 |
| Fraction 4 | 16.4 | 32.4 | 130.7 | 59.0 | balance | 5.5 |
| Fraction 5 | 17.8 | 20.9 | 87.2 | 50.9 | balance | 4.6 |
| Fraction 6 | 19.5 | 16.1 | 66.6 | 42.1 | balance | 3.0 |
| Fraction 7 | 17.4 | 9.9 | 61.6 | 28.9 | balance | 2.1 |
| Fraction 8 | 7.9 | 158.4 | balance | 0.4 | 38.5 | 0.0 |
| (aqueous) | | | | | | |
| Fraction 8 | | 7.9 | 59.3 | 22.6 | balance | 3.3 |
| (organic) | | | | | | |
| Pot | 387.5 | 0 | 0 | 0 | 0 | 0 |

A third sample of reactor crude product was distilled, and distillates were recovered across eight fractions. The fractions showed the following compositions in g/L:

TABLE 6
Example 7 Sample 3 Fractions.

| | Fraction | | | | Aceto- | 3-methyl-2- |
|---|---|---|---|---|---|---|
| Sample | Mass | NH₃ | H₂O | Acetone | nitrile | butenenitrile |
| Fraction 1 | 17.5 | 66.8 | 207.4 | 54.0 | balance | 5.6 |
| Fraction 2 | 17.9 | 40.0 | 132.8 | 52.8 | balance | 4.2 |
| Fraction 3 | 16.9 | 50.6 | 159.9 | 88.3 | balance | 6.6 |
| Fraction 4 | 17.3 | 30.0 | 124.4 | 62.2 | balance | 4.1 |
| (organic) | | | | | | |
| Fraction 5 | 16.1 | 23.8 | 91.8 | 71.6 | balance | 3.7 |
| (organic) | | | | | | |
| Fraction 6 | 22.7 | 13.5 | 66.7 | 62.7 | balance | 2.4 |
| (organic) | | | | | | |
| Fraction 7 | 18.7 | 12.4 | 74.9 | 53.5 | balance | 2.4 |
| (organic) | | | | | | |
| Fraction 8 | 9.3 | 32.5 | 111.0 | 28.9 | balance | 2.6 |
| (organic) | | | | | | |
| Fraction 4 | | 129.2 | balance | 7.0 | 83.7 | 1.2 |
| (aqueous) | | | | | | |
| Fraction 5 | | 141.4 | balance | 4.6 | 56.3 | 2.6 |
| (aqueous) | | | | | | |
| Fraction 6 | | 144.6 | balance | 2.0 | 40.0 | 0.0 |
| (aqueous) | | | | | | |
| Fraction 7 | | 147.7 | balance | 1.7 | 37.3 | 0.0 |
| (aqueous) | | | | | | |
| Fraction 8 | | 150.0 | balance | 1.6 | 37.7 | 0.0 |
| (aqueous) | | | | | | |
| Pot | 381.8 | 9.6 | balance | 1.2 | 6.1 | 0.0 |

In Tables 4-6, exhibiting various fractions of reactor crude product, pot refers to the distillation bottoms, while fraction mass refers to the total distillate fraction, and includes the combined organic and aqueous phases.

### Example 8. Pilot Scale Reactor Sample Purification

In Example 8, reactor crude product was distilled across four batches. During the distillation, ammonium bicarbonate precipitated in the condensers, requiring periodic rinsing. The released ammonia was passed through a 20 L scrubber vessel filled with roughly 15 L water. In total, this crude product was estimated to contain 5.5 kg acetonitrile, recovered in the following fractions:

TABLE 7
Example 8 glassware testing.

| | | Total ACN | Total ACN | Total ACN | | |
|---|---|---|---|---|---|---|
| | Total L | recovered in | recovered in | recovered | Total | Total |
| Glassware | NH₃ | distillates | bicarb rinses | in scrubber | ACN left | ACN |
| Run | distillates | (g) | (g) | (g) | in pot (g) | (g) |
| Test 5 (10 L) | 1.855 | 666.1 | 0 | 0 | 94.4 | 760.6 |
| Test 6 (20 L) | 4.164 | 1146.7 | 226.3 | 229.0 | 151.6 | 1753.6 |
| Test 7 (20 L) | 3.635 | 1233.9 | 971.6 | 292.5 | 229.8 | 2727.9 |
| Test 8 (~3 L) | 0.375 | 117.8 | 0 | 68.5 | 97.8 | 284.2 |
| Total (~53 L) | 10.029 | 3164.6 | 1197.9 | 590.1 | 573.8 | 5526.5 |

In Table 7, “distillates” refers to distillate fractions collected in the distillation receiver, “bicarb rinses” refers to acetonitrile recovered while flowing water through condensers to remove accumulated ammonium bicarbonate, “scrubber” refers to acetonitrile that did not condense in the distillate condensers but was captured in a separate water scrubber, and “pot” refers to the distillation bottoms.

One batch of the distillates were neutralized with 75 wt % sulfuric acid (e.g. H₂SO₄), resulting in a final pH of 3.3. Decanting the liquid from the precipitated ammonium sulfate yielded liquid which was distilled. Distillates were collected across 11 fractions, with the following compositions in g/L.

TABLE 8
Fractions in Example 8.

| | Fraction | | | | Aceto- | 3-methyl-2- |
|---|---|---|---|---|---|---|
| Sample | Mass | NH3 | H2O | Acetone | nitrile | butenenitrile |
| Fraction 1 | 30.8 | 0.0 | 99.4 | 97.4 | balance | 0.5 |
| Fraction 2 | 29.6 | 0.0 | 104.2 | 89.4 | balance | 0.7 |
| Fraction 3 | 30.1 | 0.0 | 102.6 | 77.3 | balance | 0.5 |
| Fraction 4 | 30.0 | 0.0 | 101.8 | 66.2 | balance | 0.6 |
| Fraction 5 | 29.5 | 0.0 | 103.0 | 56.8 | balance | 0.7 |
| Fraction 6 | 29.7 | 0.0 | 104.6 | 46.0 | balance | 0.9 |
| Fraction 7 | 29.7 | 0.0 | 109.0 | 37.1 | balance | 1.3 |
| Fraction 8 | 29.7 | 0.0 | 108.2 | 29.5 | balance | 1.5 |
| Fraction 9 | 30.3 | 0.0 | 108.7 | 22.0 | balance | 1.6 |
| Fraction 10 | 30.1 | 0.0 | 110.2 | 14.7 | balance | 2.2 |
| Fraction 11 | 30.9 | 0.0 | 126.8 | 7.2 | balance | 3.5 |
| Pot | 214.5 | 9.3 | balance | 1.4 | 24.3 | 0.0 |
| (aqueous) | | | | | | |
| Pot | | 0.0 | 83.3 | 2.4 | balance | 30.3 |
| (organic) | | | | | | |

Three additional batches of approximately 3 L were neutralized by dripping 75 wt H₂SO₄ into the distillates while stirring:

TABLE 9
Additional batches in Example 8.

| | | | | | | Total |
|---|---|---|---|---|---|---|
| | Sample | Sample | | 75 wt % | | time to |
| | Mass | Volume | Initial | H2SO4 | Final | add acid |
| Sample | (g) | (L) | pH | (g) | pH | (min) |
| 1 | 2730.2 | 3.2 | 12.2 | 1361.5 | 5.0 | 206 |
| 2 | 2498.4 | 2.9 | 11.7 | 1199.2 | 5.0 | 345 |
| 3 | 1759.8 | 2.1 | 11.5 | 830.4 | 5.0 | 196 |

These neutralized distillates were decanted to separate the liquid phase from the precipitated ammonium sulfate and distilled in a 5 L jacketed flask in three batches. In total 3629.0 g of distillates were recovered.

TABLE 10
Neutralized distillates in Example 8.

| | Sample | Combined | Pot | |
|---|---|---|---|---|
| | Mass | distillate | Holdup | Mass |
| Sample | (g) | mass (g) | (g) | balance |
| 1 | 1277.7 | 251.3 | 1031.4 | 1.00 |
| 2 | 2588.2 | 2081.8 | 364.6 | 0.94 |
| 3 | 3669.2 | 1295.9 | 2366.6 | 1.00 |
| Total | 7535.1 | 3629.0 | 3762.6 | 0.98 |

These distillates were combined, analyzed by GC, and found to have the following composition, in g/L:

TABLE 11
Combined distillates in Example 8.

| | | | | Aceto- | 3-methyl-2- | |
|---|---|---|---|---|---|---|
| Sample | NH₃ | H₂O | Acetone | nitrile | butenenitrile | Mesitylene |
| Combined | 0.0 | 122.0 | 47.0 | balance | 2.4 | 0.1 |
| Distillates | | | | | | |

The estimated acetonitrile content was 3268.1 g, indicating 7500 recovery of the estimated original 4362.5 g available, through these first two purification steps.

Of the 3629.0 g distillates available, 3129.4 g were basified by potassium hydroxide (eg. KOH), oxidized by potassium permanganate (e.g. KMnO₄) across, quenched with sodium thiosulfate (e.g. Na₂S₂O₃), and neutralized with 75 wt % H₂SO₄ across seven batches:

TABLE 12
Oxidized batches in Example 8.

| | g | g | g | g | g | g 75 wt % |
|---|---|---|---|---|---|---|
| Batch | distillates | H₂O | KOH | KMnO₄ | Na₂S₂O₃ | H2SO4 |
| 1 | 392.4 | 2244.7 | 42.5 | 260.2 | 6.2 | 56.8 |
| 2 | 399.6 | 2234.6 | 49.2 | 271.9 | 11.7 | 57.9 |
| 3 | 442.8 | 2371.7 | 54.5 | 292.7 | 7.3 | 66.7 |
| 4 | 532.0 | 2306.9 | 5.1 | 340.9 | 9.2 | 91.0 |
| 5 | 546.6 | 2338.9 | 62.2 | 379.9 | 7.9 | 78.5 |
| 6 | 436.0 | 2321.7 | 50.0 | 292.1 | 7.8 | 75.2 |
| 7 | 380.0 | 2227.1 | 50.9 | 255.3 | 7.8 | 62.4 |
| Total | 3129.4 | 16045.6 | 314.4 | 2093.0 | 57.9 | 488.5 |

These samples had an average approximate starting acetone concentration of 7.6 g/L. The liquid phase was decanted off of the precipitated manganese dioxide (e.g. MnO₂), filtered through a 1 um filter, and pumped into a 50 L glass vessel for distillation. The combined samples had a total volume of approximately 19 L and 0.004 g/L acetone (0.07 g total), indicating approximately 99.95% conversion of acetone in this step. The combined samples also had approximately 87.5 g/L acetonitrile (1695.3 g total) available, indicating 54% acetonitrile recovery through the KMnO₄ step.

The combined samples were distilled in the 50 L vessel and the distillate was collected in six fractions, totaling 3969.5 g, and containing cumulatively 1427.8 g acetonitrile. The distillation pot contained another 55 g acetonitrile, indicating 87% total acetonitrile recovery during the distillation, and 84% recovery in the distillate fractions. The distillate fractions were combined, basified with 20.7 g KOH, oxidized with 4.1 g KMnO₄ at 40° C. for 10 h, quenched with 4.0 g Na₂S₂O₃, and neutralized with 46.8 g of 75 wt % H₂SO₄. During the reaction, the acetone concentration decreased from 24 ppm to 3 ppm. The decrease was initially rapid, but stalled when the pH dropped from 12.2 to 11.8.

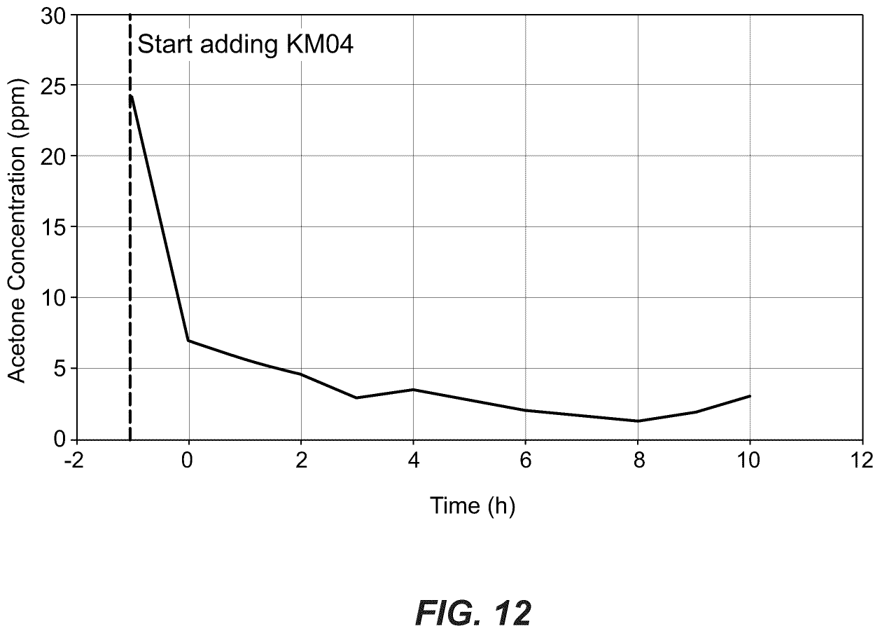

Shown in FIG. 12, the same solution was re-basified with 30.0 g KOH and re-oxidized with 8.5 g KMnO₄ for 1 h, at which point acetone was undetectable by GC. The reaction was quenched with 4.0 g Na₂S₂O₃ and neutralized with 36.5 g of 75 wt % H₂SO₄, then distilled. A total of 2042 g distillates were collected across 20 fractions. Fractions 1-15 contained most of the acetonitrile and were combined and dehydrated by salting out with potassium carbonate (e.g. K₂CO₃). The combined fractions contained approximately 183 g/L H₂O, with the balance acetonitrile.

The salting out was completed in two rounds across four parallel batches of ˜500 mL each. The organic phase comprised on composite approximately 83% of the total mass. GC analysis showed the organic phase to contain approximately 20 g/L H₂O and 0.1 g/L propionitrile, with the balance acetonitrile.

TABLE 13
Example 8 results.

| | Sample | K₂CO₃, | | | K₂CO₃, |
|---|---|---|---|---|---|
| | Mass | 1^{st} rd. | Organic phase | | 2^{nd} rd. |
| Sample | (g) | (g) | mass (g) | Organic phase % | (g) |
| 1 | 448.1 | 157.0 | 359.5 | 80.2% | 10.7 |
| 2 | 432.0 | 151.0 | 370.4 | 85.8% | 12.9 |
| 3 | 440.8 | 160.2 | 365.4 | 82.9% | 13.3 |
| 4 | 480.5 | 165.4 | 393.7 | 81.9% | 15.9 |
| Total | 1801.3 | 633.6 | 1489.1 | 82.7% | 52.8 |

The organic phases were combined and distilled in a 5 L jacketed vessel in batch. 1403.7 g liquid (approximately 1361.6 g acetonitrile, indicating 95.4% acetonitrile recovery) were decanted off the excess K₂CO₃. 1346.9 g distillates were collected, plus 37.7 g liquid was recovered from the pot, indicating approximately 98.6% recovery during this distillation (96.0% in the distillates). The distillate fractions were combined to produce a solution that was approximately 20 g/L H₂O and 0.1 g/L propionitrile, with the balance acetonitrile.

The combined distillates were purified by passing through an activated carbon column. The bulk composition of the column effluent was the same as the inlet (H₂O and propionitrile were not adsorbed on the column), but the UV absorbance decreased significantly.

The effluent of the activated carbon column was then passed through columns of 3A molecular sieves and neutral activated alumina, and finally distilled again, resulting in a purified acetonitrile product that had UV absorbance, water content, and GC purity comparable to commercially available HPLC-grade acetonitrile.

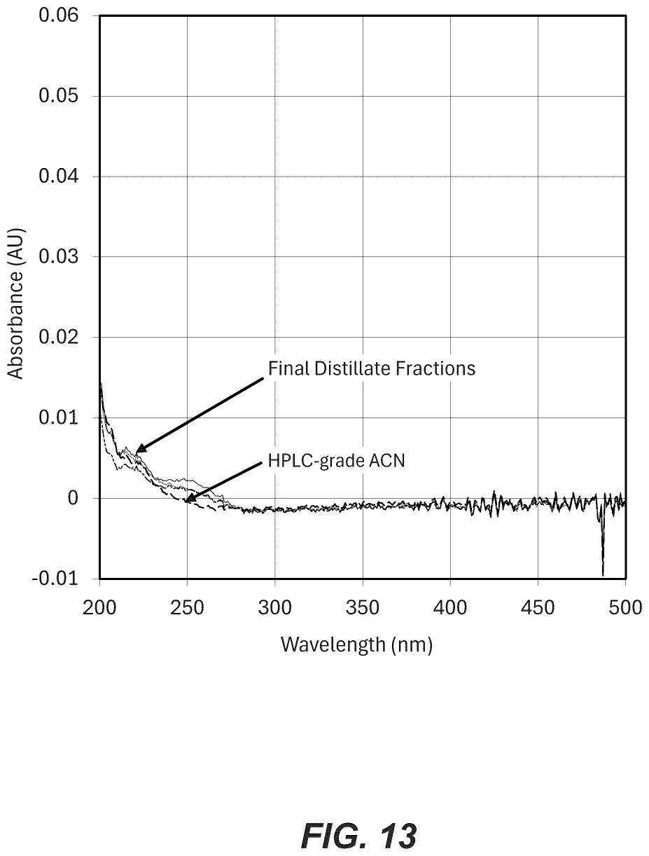

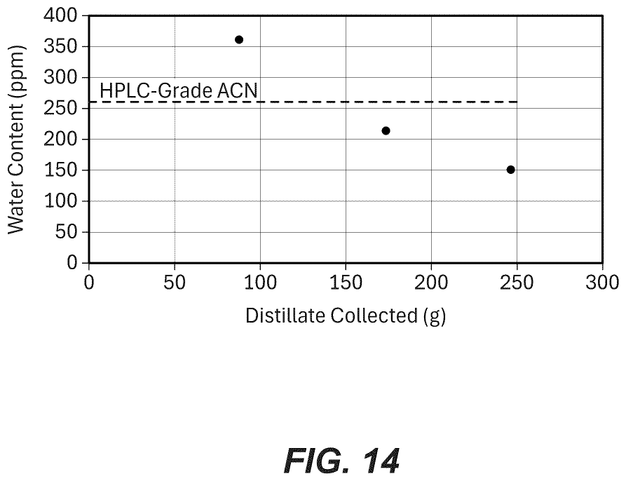

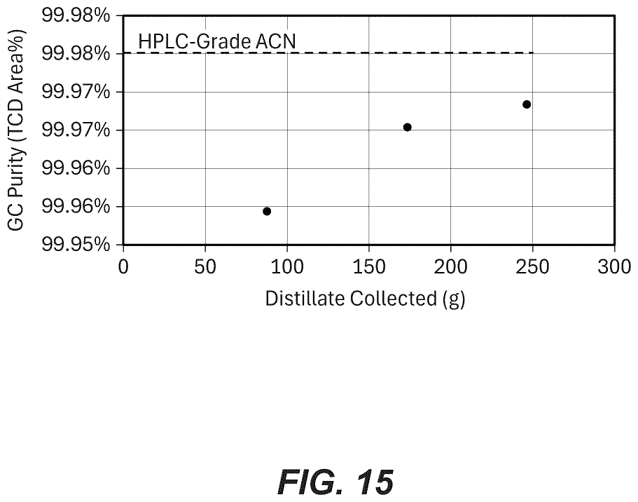

The results of Example 8 are depicted in FIGS. 13-15. The effluent of the activated carbon column was then passed through columns of 3A molecular sieves and neutral activated alumina, and finally distilled again, resulting in a purified acetonitrile product that had UV absorbance, water content, and GC purity comparable to commercially available HPLC-grade acetonitrile.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the order of which is not to be construed as designating levels of importance:

Example 1 can include a method of making an acetonitrile composition, the method comprising: (a) providing a biologically produced acetonitrile precursor; (b) purifying the biologically produced acetonitrile precursor; (c) reacting the biologically produced acetonitrile precursor with a nitrogen source and a catalyst to provide crude acetonitrile; and (d) purifying the crude acetonitrile to provide the acetonitrile composition, wherein the reacting produces no more than 20 wt % of organic products other than acetonitrile, and wherein the acetonitrile composition comprises less than 2 wt % of impurities.

Example 2 can include a method of making an acetonitrile composition, the method comprising: (a) providing a biologically produced acetonitrile precursor; (b) purifying the biologically produced acetonitrile precursor; (c) reacting the biologically produced acetonitrile precursor with a nitrogen source and a catalyst to provide crude acetonitrile; and (d) purifying the crude acetonitrile to provide the acetonitrile composition, wherein the method does not generate hydrogen cyanide.

Example 3 can include the method of example 2, wherein the acetonitrile composition comprises less than 2 wt % of impurities.

Example 4 can include the method of any one of the preceding examples, wherein the reacting produces no more than 10 wt % of organic products other than acetonitrile.

Example 5 can include the method of any one of the preceding examples, wherein the acetonitrile composition comprises less than 1 wt % of impurities.

Example 6 can include the method of any one of the preceding examples, wherein the acetonitrile composition comprises less than 0.5 wt % of impurities.

Example 7 can include the method of any one of the preceding examples, wherein the acetonitrile composition comprises less than 0.1 wt % of impurities.

Example 8 can include the method of any one of the preceding examples, wherein the wt % of impurities is measured by gas chromatography.

Example 9 can include the method of any one of the preceding examples, wherein the acetonitrile composition comprises an absorbance of less than 0.1 absorbance units (AU) at a wavelength of 200 nm.

Example 10 can include the method of any one of the preceding examples, wherein the acetonitrile composition comprises an absorbance of less than 0.05 absorbance units (AU) at a wavelength of 200 nm.

Example 11 can include the method of any one of the preceding examples, wherein the acetonitrile composition comprises an absorbance of less than 0.1 AU at a wavelength of 210 nm.

Example 12 can include the method of any one of the preceding examples, wherein the acetonitrile composition comprises an absorbance of less than 0.03 AU at a wavelength of 210 nm.

Example 13 can include the method of any one of the preceding examples, wherein the acetonitrile composition comprises an absorbance of less than 0.05 AU at a wavelength of 220 nm.

Example 14 can include the method of any one of the preceding examples, wherein the acetonitrile composition comprises an absorbance of less than 0.02 AU at a wavelength of 220 nm.

Example 15 can include the method of any one of the preceding examples, wherein the acetonitrile composition comprises an absorbance of less than 0.05 AU at a wavelength of 230 nm.

Example 16 can include the method of any one of the preceding examples, wherein the acetonitrile composition comprises an absorbance of less than 0.01 AU at a wavelength of 230 nm.

Example 17 can include the method of any one of the preceding examples, wherein the acetonitrile composition comprises an absorbance of less than 0.01 AU at a wavelength of 240 nm.

Example 18 can include the method of any one of the preceding examples, wherein the acetonitrile composition comprises an absorbance of less than 0.005 AU at a wavelength of 240 nm.

Example 19 can include the method of any one of the preceding examples, wherein the acetonitrile composition comprises an absorbance of less than 0.01 AU at a wavelength of 260 nm.

Example 20 can include the method of any one of the preceding examples, wherein the acetonitrile composition comprises an absorbance of less than 0.005 AU at a wavelength of 260 nm.

Example 21 can include the method of any one of the preceding examples, wherein the acetonitrile composition comprises an absorbance of less than 0.01 AU at a wavelength of 280 nm.

Example 22 can include the method of any one of the preceding examples, wherein the acetonitrile composition comprises an absorbance of less than 0.005 AU at a wavelength of 280 nm.

Example 23 can include the method of any one of the preceding examples, wherein the acetonitrile composition comprises an absorbance of less than 0.01 AU at a wavelength of 400 nm.

Example 24 can include the method of any one of the preceding examples, wherein the acetonitrile composition comprises an absorbance of less than 0.005 AU at a wavelength of 400 nm.

Example 25 can include the method of any one of the preceding examples, wherein the acetonitrile composition comprises no more than 20 ppm water.

Example 26 can include the method of any one of the preceding examples, wherein the acetonitrile composition comprises no more than 10 ppm water.

Example 27 can include the method of any one of the preceding examples, wherein the acetonitrile composition is anhydrous acetonitrile.

Example 28 can include the method of any one of the preceding examples, wherein the biologically produced acetonitrile precursor is produced by fermentation.

Example 29 can include the method of any one of the preceding examples, wherein the biologically produced acetonitrile precursor is produced by bacterial fermentation.

Example 30 can include the method of any one of the preceding examples, wherein the biologically produced acetonitrile precursor is produced by aerobic bacterial fermentation.

Example 31 can include the method of any one of the preceding examples, wherein the purifying the biologically produced acetonitrile precursor comprises liquid-liquid extraction, distillation, or a combination thereof.

Example 32 can include the method of example 31, wherein the liquid-liquid extraction comprises contacting the biologically produced acetonitrile precursor with an organic solvent.

Example 33 can include the method of example 32, wherein the organic solvent is ethyl acetate, butyl acetate, diethyl ether, dichloromethane, toluene, chloroform, methyl tert-butyl ether, toluene, chloroform, hexane, benzene, acetone, or a combination thereof.

Example 34 can include the method of any one of the preceding examples, wherein the biologically produced acetonitrile precursor is acetic acid.

Example 35 can include the method of any one of the preceding examples, wherein the biologically produced acetonitrile precursor is glacial acetic acid.

Example 36 can include the method of any one of the preceding examples, wherein the catalyst is a heterogeneous catalyst.

Example 37 can include the method of any one of the preceding examples, wherein the catalyst is aluminum oxide, titanium dioxide, zirconium dioxide, or tungsten oxide or a combination thereof.

Example 38 can include the method of any one of the preceding examples, wherein the catalyst is a transition metal oxide or a combination of transition metal oxides.

Example 39 can include the method of example 38, wherein the transition metal oxide is zirconium dioxide, tungsten oxide, or a combination thereof.

Example 40 can include the method of any one of the preceding examples, wherein the reacting comprises heating to a temperature of no more than 350° C.

Example 41 can include the method of any one of the preceding examples, wherein the reacting comprises heating to a temperature of from about 250° C. to about 350° C.

Example 42 can include the method of any one of the preceding examples, wherein the nitrogen source is ammonia.

Example 43 can include the method of any one of the preceding examples, wherein the reacting comprises an absolute pressure of from about 0.1 atm to about 20 atm.

Example 44 can include the method of any one of the preceding examples, wherein the reacting comprise a pressure of from about 1 atm to about 4 atm.

Example 45 can include the method of any one of the preceding examples, wherein the purifying the crude acetonitrile comprises distillation, oxidation, polishing, or a combination thereof.

Example 46 can include the method of example 45, wherein the distillation removes excess nitrogen source.

Example 47 can include the method of example 46, wherein the excess nitrogen source is recycled in (c).

Example 48 can include the method of any one of the preceding examples, wherein the purifying comprises use of an oxidant.

Example 49 can include the method of any one of example 48, wherein the oxidant is a Lewis acid.

Example 50 can include the method of any one of example 48 or 49, wherein the oxidant is potassium permanganate, ozone, hydrogen peroxide, potassium superoxide, or an oxidizing agent generated via UV light.

Example 51 can include the method of any one of examples 48-50, wherein the oxidant is potassium permanganate.

Example 52 can include the method of any one of the preceding examples, wherein the purifying the crude acetonitrile is completed in acidic, neutral, or basic conditions.

Example 53 can include the method of any one of the preceding examples, wherein the purifying the crude acetonitrile is completed in basic conditions.

Example 54 can include the method of any examples 45-53, wherein the polishing of the crude acetonitrile comprises contacting the crude acetonitrile with a zeolite, ion-exchange resin, activated alumina, activated carbon, or a combination thereof.

Example 55 can include the method of any examples 45-54, wherein the polishing of the crude acetonitrile comprises contacting the crude acetonitrile with silica, calcium sulfate, molecular sieves, a zeolite, alumina, or magnesium sulfate.

Example 56 can include the method of any one of the preceding examples, wherein the method produces the acetonitrile composition in a quantity of at least 100 L.

Example 57 can include the method of any one of the preceding examples, wherein the method produces the acetonitrile composition in a quantity of at least 150 L.

Example 58 can include the method of any one of the preceding examples, wherein the acetonitrile composition is an oligonucleotide synthesis grade, ACS grade, HPLC grade, LC/MS grade, or pharmaceutical grade acetonitrile composition.

Example 59 can include the method of any one of examples 1-57, wherein the acetonitrile composition is an oligonucleotide synthesis grade acetonitrile composition.

Example 60 can include the method of any one of the preceding examples, wherein the method does not produce acrylonitrile.

Example 61 can include the method of any one of the preceding examples, wherein the method does not comprise use of an acetonitrile precursor derived from a fossil fuel.

Example 62 can include the method of any one of the preceding examples, wherein the acetonitrile composition comprises a reduction in carbon intensity (CI) as compared to an acetonitrile composition produced using the SOHIO process.

Example 63 can include the method of any one of the preceding examples, wherein the acetonitrile composition comprises an about 90% reduction in CI as compared to an acetonitrile composition produced using the SOHIO process.

Example 64. A composition comprising acetonitrile, produced by the method of any one of the preceding examples.

Example 65 can include the composition of example 64, wherein the composition comprises: at least 98 wt % acetonitrile derived from a biologically produced precursor and less than 10 ppm water and does not comprise acetonitrile derived from a fossil fuel source.

Example 66 can include the composition of example 65, wherein the composition comprises at least 99 wt % acetonitrile derived from a biologically produced precursor.

Example 67 can include the composition of example 65 or 66, wherein the composition comprises at least 99.5 wt % acetonitrile derived from a biologically produced precursor.

Example 68 can include the composition of any one of examples 64-67, wherein the composition comprises at least 99.9 wt % acetonitrile derived from a biologically produced precursor.

Example 69 can include the composition of any one of examples 64-68, wherein the composition comprises an absorbance of less than 0.1 absorbance units (AU) at a wavelength of 200 nm.

Example 70 can include the composition of any one of examples 64-69, wherein the composition comprises an absorbance of less than 0.05 absorbance units (AU) at a wavelength of 200 nm.

Example 71 can include the composition of any one of examples 64-70, wherein the composition comprises an absorbance of less than 0.1 AU at a wavelength of 210 nm.

Example 72 can include the composition of any one of examples 64-71, wherein the composition comprises an absorbance of less than 0.03 AU at a wavelength of 210 nm.

Example 73 can include the composition of any one of examples 64-72, wherein the composition comprises an absorbance of less than 0.05 AU at a wavelength of 220 nm.

Example 74 can include the composition of any one of examples 64-73, wherein the composition comprises an absorbance of less than 0.02 AU at a wavelength of 220 nm.

Example 75 can include the composition of any one of examples 64-74, wherein the composition comprises an absorbance of less than 0.05 AU at a wavelength of 230 nm.

Example 76 can include the composition of any one of examples 64-75, wherein the composition comprises an absorbance of less than 0.01 AU at a wavelength of 230 nm.

Example 77 can include the composition of any one of examples 64-76, wherein the composition comprises an absorbance of less than 0.01 AU at a wavelength of 240 nm.

Example 78 can include the composition of any one of examples 64-77, wherein the composition comprises an absorbance of less than 0.005 AU at a wavelength of 240 nm.

Example 79 can include the composition of any one of examples 64-78, wherein the composition comprises an absorbance of less than 0.01 AU at a wavelength of 260 nm.

Example 80 can include the composition of any one of examples 64-79, wherein the composition comprises an absorbance of less than 0.005 AU at a wavelength of 260 nm.

Example 81 can include the composition of any one of examples 64-80, wherein the composition comprises an absorbance of less than 0.01 AU at a wavelength of 280 nm.

Example 82 can include the composition of any one of examples 64-81, wherein the composition comprises an absorbance of less than 0.005 AU at a wavelength of 280 nm.

Example 83 can include the composition of any one of examples 64-82, wherein the composition comprises an absorbance of less than 0.01 AU at a wavelength of 400 nm.

Example 84 can include the composition of any one of examples 64-83, wherein the composition comprises an absorbance of less than 0.005 AU at a wavelength of 400 nm.

Example 85 can include the composition of any one of examples 64-84, wherein the composition comprises anhydrous acetonitrile.

We claim:

1. A method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition, wherein the reacting produces no more than 20 wt % of organic products other than acetonitrile, and
wherein the acetonitrile composition comprises less than 2 wt % of impurities.

2. The method of claim 1, wherein the acetonitrile composition comprises less than 2 wt % of impurities.

3. The method of claim 1, wherein the acetonitrile composition comprises less than 1 wt % of impurities.

4. The method of claim 1, wherein the acetonitrile composition comprises less than 0.5 wt % of impurities.

5. The method of claim 1, wherein the acetonitrile composition comprises less than 0.1 wt % of impurities.

6. The method of claim 1, wherein the reacting produces no more than 10 wt % of organic products other than acetonitrile.

7. The method of claim 1, wherein the biologically produced acetonitrile precursor is produced by fermentation, and wherein the purifying the biologically produced acetonitrile precursor comprises liquid-liquid extraction, distillation, or a combination thereof.

8. The method of claim 1, wherein the reacting comprises heating to a temperature of no more than 350° C.

9. The method of claim 1, wherein the reacting comprises an absolute pressure of from about 0.1 atm to about 20 atm.

10. The method of claim 1, wherein the purifying the crude acetonitrile comprises distillation, oxidation, polishing, or a combination thereof.

11. The method of claim 1, wherein the acetonitrile composition is a oligonucleotide synthesis grade acetonitrile composition or pharmaceutical grade acetonitrile composition.

12. A method of making an acetonitrile composition, the method comprising:
(a) providing a biologically produced acetonitrile precursor;
(b) purifying the biologically produced acetonitrile precursor;
(c) reacting the biologically produced acetonitrile precursor with a nitrogen source and a catalyst to provide crude acetonitrile; and
(d) purifying the crude acetonitrile to provide the acetonitrile composition,
wherein the method does not generate hydrogen cyanide.

13. The method of claim 12, wherein the method does not produce acrylonitrile.

14. The method of claim 12, wherein the method does not comprise use of an acetonitrile precursor derived from a fossil fuel.

15. The method of claim 12, wherein the acetonitrile composition comprises a reduction in carbon intensity (CI) as compared to an acetonitrile composition produced using the SOHIO process.

16. A composition comprising acetonitrile, produced by the method of claim 1.

17. The composition of claim 16, wherein the composition comprises: at least 98 wt % acetonitrile derived from a biologically produced precursor and less than 10 ppm water and does not comprise acetonitrile derived from a fossil fuel source.

18. The composition of claim 17, wherein the composition comprises at least 99 wt % acetonitrile derived from a biologically produced precursor.

19. The composition of claim 16, wherein the composition comprises an absorbance of less than 0.1 absorbance units (AU) at a wavelength any of 200 nm to 400 nm.

20. The composition of claim 16, wherein the composition comprises anhydrous acetonitrile.
